(19) United States
(12) Patent Application Publication
(10) Pub. No.: US 2026/0285825 A1
(43) Pub. Date: Sep. 24, 2026

(54) Crystalline complexes between velagliflozin and selected co-crystal forming agents, methods for their preparation and the use thereof for preparing medicaments

(71) Applicant: Boehringer Ingelheim Vetmedica GmbH, Ingelheim am Rhein (DE)

(72) Inventors: Ulrike WERTHMANN, Biberach an der Riss (DE); Alexander Werner BOEHM, Klein-Winternheim (DE); Jerome MENEGOTTO, Toulouse (FR); Franck TAILLEZ, Toulouse (FR)

(21) Appl. No.: 19/473,404

(22) PCT Filed: Apr. 18, 2024
(86) PCT No.: PCT/EP2024/060490

§ 371 Date: Oct. 7, 2025

(30) Foreign Application Priority Data
Apr. 24, 2023 (EP) 23169467.0

(51) Int. Cl. C07D 309/10; A61K 31/351; C07D 207/16; C07D 473/10

(52) U.S. Cl. CPC C07D 309/10; A61K 31/351; C07D 207/16; C07D 473/10

(57) ABSTRACT

The invention relates to crystalline complexes of velagliflozin and selected co-crystal forming agents, to methods for their preparation as well as to the use thereof for preparing medicaments.

### INCORPORATION BY REFERENCE

All references cited herein, are incorporated by reference herein, in their entirety.

### BACKGROUND OF THE INVENTION

### A. Field of the Invention

The invention relates to the field of medicine, particularly veterinary medicine. In particular, the invention relates to crystalline complexes of velagliflozin and selected co-crystal forming agents, to methods for their preparation as well as to the use thereof for preparing medicaments.

### B. Background and Description of the Related Art

The compound 1-cyano-2-(4-cyclopropyl-benzyl)-4-(β-D-glucopyranos-1-yl)-benzene (in the following referred to it by its INN “velagliflozin”) is described in WO 2007/093610 and WO 2007/128749 and has the chemical structure according to formula A:

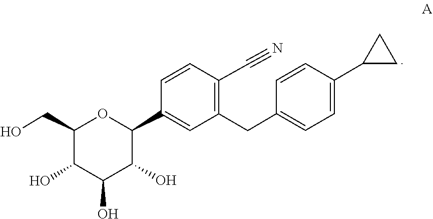 [SMILES: N#Cc1ccc([C@@H]2O[C@H](CO)[C@@H](O)[C@H](O)[C@H]2O)cc1Cc1ccc(C2CC2)cc1]

The compounds described in these two WO publications have a valuable inhibitory effect on the sodium-dependent glucose cotransporter SGLT, particularly SGLT-2. The methods of manufacture of velagliflozin as described therein do not yield crystalline forms nor do they yield crystalline complexes between velagliflozin and selected co-crystal forming agents.

A certain pharmaceutical activity is of course the basic prerequisite to be fulfilled by a pharmaceutically active agent before same is approved as a medicament on the market. However, there are a variety of additional requirements a pharmaceutically active agent has to comply with. These requirements are based on various parameters which are connected with the nature of the active substance itself. Without being restrictive, examples of these parameters are the stability of the active agent under various environmental conditions, its stability during production of the pharmaceutical formulation and the stability of the active agent in the final medicament compositions. The pharmaceutically active substance used for preparing the pharmaceutical compositions should be as pure as possible and its stability in long-term storage must be guaranteed under various environmental conditions. This is essential to prevent the use of pharmaceutical compositions which contain, in addition to the actual active substance, breakdown products thereof, for example. In such cases the content of active substance in the medicament might be less than that specified.

Uniform distribution of the medicament in the formulation is a critical factor, particularly when the medicament has to be given in low doses. To ensure uniform distribution, the particle size of the active substance can be reduced to a suitable level, e.g. by grinding. Since breakdown of the pharmaceutically active substance as a side effect of the grinding (or micronising) has to be avoided as far as possible, in spite of the hard conditions required during the process, it is essential that the active substance should be highly stable throughout the grinding process. Only if the active substance is sufficiently stable during the grinding process it is possible to produce a homogeneous pharmaceutical formulation which always contains the specified amount of active substance in a reproducible manner.

Another problem which may arise in the grinding process for preparing the desired pharmaceutical formulation is the input of energy caused by this process and the stress on the surface of the crystals. This may in certain circumstances lead to polymorphous changes, to amorphization or to a change in the crystal lattice. Since the pharmaceutical quality of a pharmaceutical formulation requires that the active substance should always have the same crystalline morphology, the stability and properties of the crystalline active substance are subject to stringent requirements from this point of view as well.

The stability of a pharmaceutically active substance is also important in pharmaceutical compositions for determining the shelf life of the particular medicament; the shelf life is the length of time during which the medicament can be administered without any risk. High stability of a medicament in the abovementioned pharmaceutical compositions under various storage conditions is therefore an additional advantage for both the patient and the manufacturer.

The absorption of moisture reduces the content of pharmaceutically active substance as a result of the increased weight caused by the uptake of water. Pharmaceutical compositions with a tendency to absorb moisture have to be protected from moisture during storage, e.g. by the addition of suitable drying agents or by storing the drug in an environment where it is protected from moisture. Preferably, therefore, a pharmaceutically active substance should be at best slightly hygroscopic.

Furthermore, the availability of a well-defined crystalline form allows the purification of the drug substance by recrystallization.

Apart from the requirements indicated above, it should be generally borne in mind that any change to the solid state of a pharmaceutical composition which is capable of improving its physical and chemical stability gives a significant advantage over less stable forms of the same medicament.

WO 2014/016381 discloses crystalline complexes of velagliflozin with in particular L-proline, preferably as hydrate form. The preferred crystalline complex described therein is a monohydrate co-crystal and characterized by a molar ratio of velagliflozin and L-proline and crystal water of 1:1:1.

WO 2023/006718 discloses the use of one or more SGLT-2 inhibitors or pharmaceutically acceptable forms thereof for the prophylaxis and/or treatment of one or more cardiac diseases in a non-human mammal/non-human mammal patient excluding a feline, in particular a canine/canine patient.

The objective of the invention is to provide new, stable crystalline forms of velagliflozin, which meet important requirements imposed on pharmaceutically active substances as those mentioned above.

### SUMMARY OF THE INVENTION

In a first aspect the present invention relates to a crystalline complex between velagliflozin and a co-crystal forming agent.

In the light of the pharmaceutical efficacy of velagliflozin and the advantageous physical chemical properties of the crystalline complex a second aspect of the present invention relates to a pharmaceutical composition or medicament comprising one or more crystalline complexes as defined hereinbefore and hereinafter and/or mixtures of crystalline forms as defined hereinbefore and hereinafter.

A further aspect of the present invention relates to the herein defined crystalline complexes or the herein defined mixtures of crystalline forms for use as a medicament.

In a further aspect the present invention relates to one or more crystalline complexes as defined hereinbefore or hereinafter and/or the pharmaceutical composition as defined hereinbefore or hereinafter and/or the medicament as defined hereinbefore or hereinafter for use in a method of treatment and/or prevention of diseases or conditions, which can be influenced by inhibiting sodium-dependent glucose cotransporter SGLT, preferably SGLT-2. A corresponding method of treatment and/or prevention of diseases or conditions, which can be influenced by inhibiting sodium-dependent glucose cotransporter SGLT, preferably SGLT-2, as well as the corresponding use for the preparation of a medicament for the prevention and/or treatment of diseases or conditions, which can be influenced by inhibiting sodium-dependent glucose cotransporter SGLT, preferably SGLT-2, are also intended to be comprised by the present invention.

In a further aspect the present invention relates to one or more crystalline complexes as defined hereinbefore or hereinafter and/or the pharmaceutical composition as defined hereinbefore or hereinafter and/or the medicament as defined hereinbefore or hereinafter for use in a method of inhibiting the sodium-dependent glucose cotransporter SGLT-2.

In a further aspect the present invention relates to a method for making one or more crystalline complexes as defined hereinbefore and hereinafter, said method comprising the following steps:
(a) preparing a solution of velagliflozin and the co-crystal forming agent in a solvent or a mixture of solvents;
(b) storing the solution to precipitate the crystalline complex out of solution;
(c) removing the precipitate from the solution; and
(d) drying the precipitate optionally until any excess of said solvent or mixture of solvents has been removed.

Further aspects of the present invention become apparent to the one skilled in the art from the following detailed description of the invention and the examples.

### BRIEF DESCRIPTION OF THE DRAWINGS

FIG. 1A shows an X-ray powder diffraction pattern of a representative batch of the crystalline complex of velagliflozin with L-proline (1:3), FORM 1000.

FIG. 1B shows the determination of the melting point via DSC and of the weight loss via TG of a representative batch of the crystalline complex of velagliflozin with L-proline (1:3), FORM 1000.

FIG. 2A shows an X-ray powder diffraction pattern of a representative batch of the crystalline complex of velagliflozin with L-proline (1:1-1:1.3), FORM 1001.

FIG. 2B shows the determination of the melting point via DSC and of the weight loss via TG of a representative batch of the crystalline complex of velagliflozin with L-proline (1:1-1:1.3), FORM 1001.

FIG. 3A shows an X-ray powder diffraction pattern of a representative batch of the crystalline complex of velagliflozin with L-proline (1:1.5), FORM 1002.

FIG. 3B shows the determination of the melting point via DSC and of the weight loss via TG of a representative batch of the crystalline complex of velagliflozin with L-proline (1:1.5), FORM 1002.

FIG. 4A shows an X-ray powder diffraction pattern of a representative batch of the crystalline complex of velagliflozin with L-proline (1:1), FORM 1003.

FIG. 4B shows the determination of the melting point via DSC and of the weight loss via TG of a representative batch of the crystalline complex of velagliflozin with L-proline (1:1), FORM 1003.

FIG. 5A shows an X-ray powder diffraction pattern of a representative batch of the crystalline complex of velagliflozin with L-proline (1:2), FORM 1004.

FIG. 5B shows the determination of the melting point via DSC and of the weight loss via TG of a representative batch of the crystalline complex of velagliflozin with L-proline (1:2), FORM 1004.

FIG. 6A shown an X-ray powder diffraction pattern of a representative batch of the crystalline complex of velagliflozin with L-proline (1:1), FORM 1005.

FIG. 7A shown an X-ray powder diffraction pattern of a representative batch of the crystalline complex of velagliflozin with L-proline (1:1.4-1:1.8), FORM 1006.

FIG. 7B shows the determination of the melting point via DSC and of the weight loss via TG of a representative batch of the crystalline complex of velagliflozin with L-proline (1:1.4-1:1.8), FORM 1006.

FIG. 8A shown an X-ray powder diffraction pattern of a representative batch of the crystalline complex of velagliflozin with L-proline (1:1), FORM 1007.

FIG. 8B shows the determination of the melting point via DSC of a representative batch of the crystalline complex of velagliflozin with L-proline (1:1), FORM 1007.

FIG. 9A shown an X-ray powder diffraction pattern of a representative batch of the crystalline complex of velagliflozin with L-proline (1:1) in tetrahydrofuran, FORM 1008.

FIG. 9B shows the determination of the melting point via DSC and of the weight loss via TG of a representative batch of the crystalline complex of velagliflozin with L-proline (1:1) in tetrahydrofuran, FORM 1008.

FIG. 10A shown an X-ray powder diffraction pattern of a representative batch of the crystalline complex of velagliflozin with theobromine (1:1), FORM 1009.

FIG. 10B shows the determination of the melting point via DSC and of the weight loss via TG of a representative batch of the crystalline complex of velagliflozin with theobromine (1:1), FORM 1009.

### DETAILED DESCRIPTION OF THE INVENTION

Surprisingly, it has been found that there exist different crystalline complexes between velagliflozin and co-crystal forming agents selected from L-proline, theobromine, 3,5-dihydroxybenzoic acid, maltol, thymol, and caffeine. Such crystalline complexes fulfill important requirements as mentioned hereinbefore. Accordingly, the present invention relates to a crystalline complex between velagliflozin and a co-crystal forming agent selected from L-proline, theobromine, 3,5-dihydroxybenzoic acid, maltol, thymol, and caffeine.

Preferred co-crystal forming agents are selected from L-proline and theobromine.

In a preferred configuration, the co-crystal forming agent is L-proline.

In another preferred configuration, the co-crystal forming agent is theobromine.

In an alternative configuration, the co-crystal forming agent is 3,5-dihydroxybenzoic acid.

In an alternative configuration, the co-crystal forming agent is maltol.

In an alternative configuration, the co-crystal forming agent is thymol.

In an alternative configuration, the co-crystal forming agent is caffeine.

According to the present invention, the molar ratio of velagliflozin and the co-crystal forming agent is in the range from about 1:1 to about 1:3.

The crystalline complexes of velagliflozin and the co-crystal forming agent may be identified and distinguished from other crystalline forms by means of their characteristic X-ray powder diffraction (XRPD) patterns.

Different co-crystals of velagliflozin with L-proline or theobromine are considered as preferred embodiments of the present invention:

In a preferred embodiment the crystalline complex is a complex between velagliflozin and L-proline and it has a content of water of about 0 mol, i.e. it is a water-free/anhydrous co-crystal of velagliflozin with L-proline.

In another preferred embodiment the crystalline complex has a content of water of about 0 mol, characterized in that the crystalline complex is a complex (1:3) between velagliflozin and L-proline; and characterized by an X-ray powder diffraction pattern that comprises peaks at 5.77, 16.89, 18.95 and 20.39 degrees 2Θ (±0.1 degrees 2Θ), wherein said X-ray powder diffraction pattern is made using CuK_{α1} radiation.

It is preferred, when said X-ray powder diffraction pattern further comprises peaks at 17.40, 21.59, 23.69 and 25.19 degrees 2Θ (±0.1 degrees 2Θ), wherein said X-ray powder diffraction pattern is made using Cuk 1 radiation.

In particular, said X-ray powder diffraction pattern further comprises peaks at 5.10, 8.06, 9.64, 18.50, 19.36, 21.10, 23.23 and 26.06 degrees 2Θ (±0.1 degrees 2Θ), wherein said X-ray powder diffraction pattern is made using CuK_{α1} radiation.

More specifically, said X-ray powder diffraction pattern further comprises peaks at 11.58, 15.24, 17.88, 19.57, 22.13, 22.76, 24.64, 27.63 and 28.22 degrees 2Θ (±0.1 degrees 2Θ), wherein said X-ray powder diffraction pattern is made using CuK_{α1} radiation.

Even more specifically, said X-ray powder diffraction pattern, made using CuK_{α1} radiation comprises peaks at degrees 2Θ (±0.1 degrees 2Θ) as contained in Table 1.

TABLE 1
X-ray powder diffraction pattern of the crystalline complex of FORM
1000 (only peaks up to 30° in 2 Θ are listed):

| | 2 Θ | Intensity I/I₀ |
|---|---|---|
| Peak No | [°] | [%] |
| 1 | 5.10 | 48.7 |
| 2 | 5.77 | 100.0 |
| 3 | 8.06 | 26.0 |
| 4 | 9.06 | 5.6 |
| 5 | 9.38 | 6.1 |
| 6 | 9.64 | 29.8 |
| 7 | 10.16 | 4.8 |
| 8 | 11.58 | 14.5 |
| 9 | 14.82 | 5.1 |
| 10 | 15.24 | 16.4 |
| 11 | 16.89 | 83.3 |
| 12 | 17.40 | 31.9 |
| 13 | 17.88 | 12.1 |
| 14 | 18.50 | 29.9 |
| 15 | 18.95 | 85.3 |
| 16 | 19.36 | 27.5 |
| 17 | 19.57 | 21.0 |
| 18 | 20.39 | 93.0 |
| 19 | 21.10 | 28.7 |
| 20 | 21.59 | 36.8 |
| 21 | 22.13 | 12.5 |
| 22 | 22.76 | 14.6 |
| 23 | 23.23 | 28.4 |
| 24 | 23.69 | 52.9 |
| 25 | 24.64 | 10.8 |
| 26 | 25.19 | 43.2 |
| 27 | 26.06 | 29.9 |
| 28 | 26.88 | 7.9 |
| 29 | 27.63 | 11.9 |
| 30 | 28.22 | 16.0 |

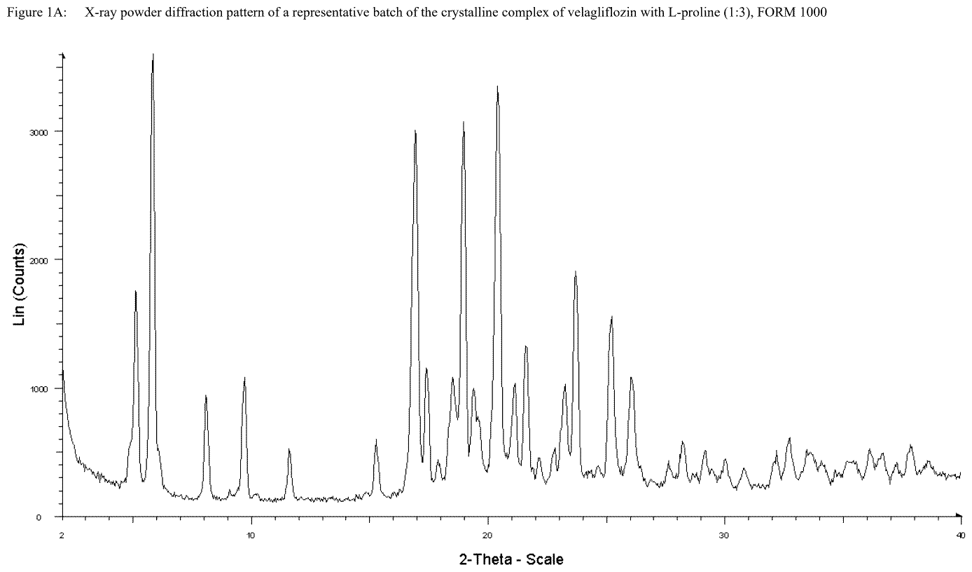

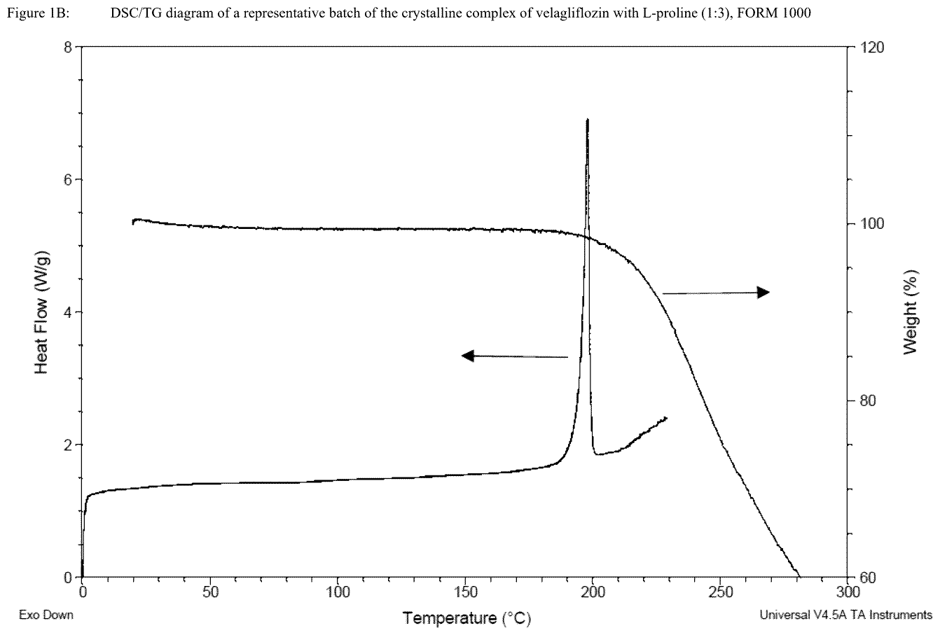

Even more specifically, said X-ray powder diffraction pattern, made using CuK_{α1} radiation, comprises peaks at degrees 2Θ (±0.1 degrees 2Θ) as shown in FIG. 1A.

Furthermore, said crystalline complex of velagliflozin with L-proline is characterized by a melting point of 195° C. (determined via DSC; evaluated as onset-temperature; heating rate 10 K/min). The obtained DSC curve is shown in FIG. 1B.

In another alternative embodiment, the crystalline complex is a complex of velagliflozin and L-proline with a molar ratio in the range from about 1:1 to about 1:1.3; and characterized by an X-ray powder diffraction pattern that comprises peaks at 4.00, 6.07, 7.30 and 20.36 degrees 2Θ (±0.1 degrees 2Θ), wherein said X-ray powder diffraction pattern is made using CuK_{α1} radiation.

It is preferred, when said X-ray powder diffraction pattern further comprises peaks at 18.29, 18.70, 19.90 and 22.09 degrees 2Θ (±0.1 degrees 2Θ), wherein said X-ray powder diffraction pattern is made using CuK_{α1} radiation.

In particular, said X-ray powder diffraction pattern further comprises peaks at 14.59, 16.84, 17.24, 23.39, 24.49, 25.62, 27.10, and 27.45 degrees 2Θ (±0.1 degrees 2Θ), wherein said X-ray powder diffraction pattern is made using CuK_{α1} radiation.

More specifically, said X-ray powder diffraction pattern further comprises peaks at 16.15, 22.57, 24.28, 24.74, 29.45, 30.79, 34.97 and 36.18 degrees 2Θ (±0.1 degrees 2Θ), wherein said X-ray powder diffraction pattern is made using CuK_{α1} radiation.

Even more specifically, said X-ray powder diffraction pattern, made using CuK_{α1} radiation comprises peaks at degrees 2Θ (±0.1 degrees 2Θ) as contained in Table 2.

TABLE 2
X-ray powder diffraction pattern of
the crystalline complex of FORM 1001:

| | 2 Θ | Intensity I/I₀ |
|---|---|---|
| Peak No | [°] | [%] |
| 1 | 4.00 | 100.0 |
| 2 | 6.07 | 20.1 |
| 3 | 7.30 | 21.6 |
| 4 | 12.10 | 4.1 |
| 5 | 12.82 | 3.5 |
| 6 | 14.59 | 6.6 |
| 7 | 16.15 | 5.4 |
| 8 | 16.84 | 5.6 |
| 9 | 17.24 | 9.3 |
| 10 | 18.29 | 11.2 |
| 11 | 18.70 | 14.9 |
| 12 | 19.90 | 10.8 |
| 13 | 20.36 | 59.2 |
| 14 | 22.09 | 10.0 |
| 15 | 22.57 | 5.2 |
| 16 | 23.39 | 8.9 |
| 17 | 24.28 | 5.5 |
| 18 | 24.49 | 6.9 |
| 19 | 24.74 | 5.3 |
| 20 | 25.62 | 6.7 |
| 21 | 27.10 | 6.0 |
| 22 | 27.45 | 7.7 |
| 23 | 29.45 | 5.3 |
| 24 | 30.79 | 4.4 |
| 25 | 31.66 | 4.1 |
| 26 | 32.94 | 3.6 |
| 27 | 33.25 | 3.8 |
| 28 | 34.97 | 4.6 |
| 29 | 35.68 | 4.0 |
| 30 | 36.18 | 4.2 |

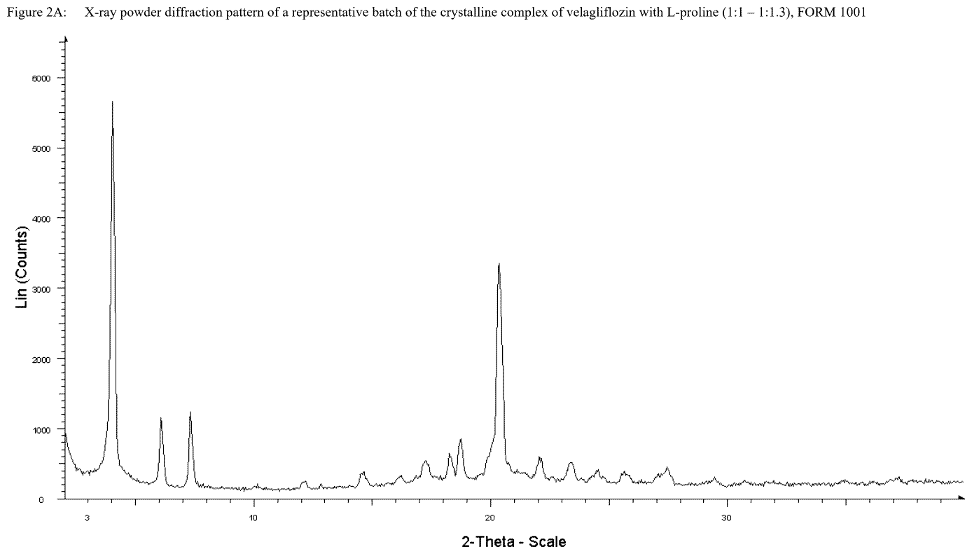

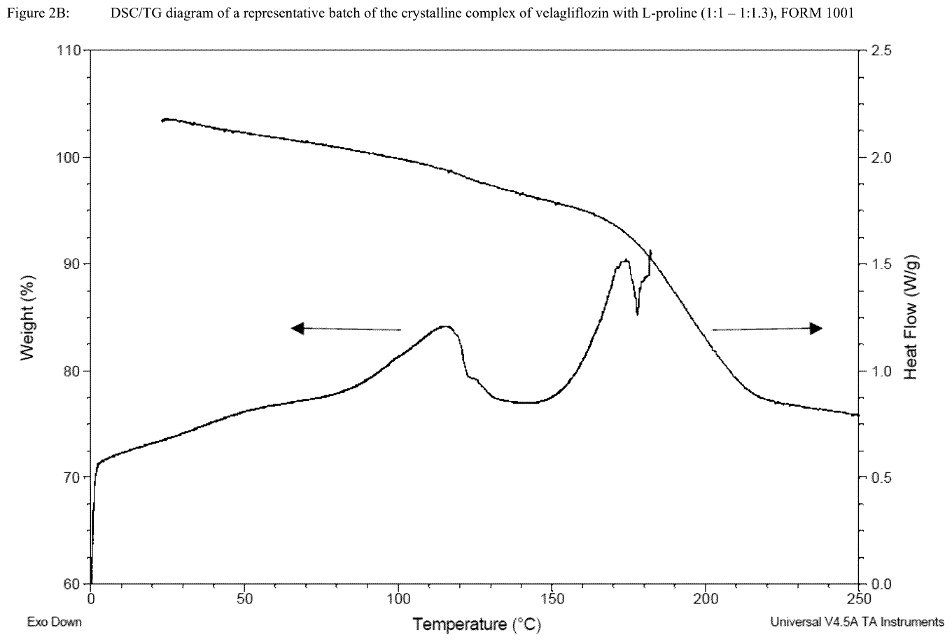

Even more specifically, said X-ray powder diffraction pattern, made using CuK_{α1} radiation, comprises peaks at degrees 2Θ (±0.1 degrees 2Θ) as shown in FIG. 2A.

Said crystalline complex of velagliflozin with L-proline shows a weight loss by thermal gravimetry (TG). The observed weight loss indicates that the crystalline form contains water which may be bound by adsorption and/or may be part of the crystalline lattice, i.e the crystalline form may be present as a crystalline hydrate.

Furthermore, said crystalline complex of velagliflozin with L-proline is characterized by a dehydration noted around 80° C. (determined via DSC; evaluated as onset-temperature; heating rate 10 K/min). The obtained DSC curve is shown in FIG. 2B.

In another alternative embodiment, the crystalline complex is a complex (1:1.5) between velagliflozin and L-proline; and characterized by an X-ray powder diffraction pattern that comprises peaks at 7.47, 16.28, 18.87 and 25.84 degrees 2Θ (±0.1 degrees 2Θ), wherein said X-ray powder diffraction pattern is made using CuK_{α1} radiation.

It is preferred, when said the X-ray powder diffraction pattern further comprises peaks at 5.47, 10.57, 20.51 and 20.65 degrees 2Θ (±0.1 degrees 2Θ), wherein said X-ray powder diffraction pattern is made using CuK_{α1} radiation.

In particular, said X-ray powder diffraction pattern further comprises peaks at 11.59, 17.20, 17.40, 21.24, 21.63, 21.96, 22.53 and 23.17 degrees 2Θ (±0.1 degrees 2Θ), wherein said X-ray powder diffraction pattern is made using CuK_{α1} radiation.

More specifically, said X-ray powder diffraction pattern further comprises peaks at 5.75, 10.91, 15.59, 19.37, 19.73, 19.86, 24.25 and 24.89 degrees 2Θ (±0.1 degrees 2Θ), wherein said X-ray powder diffraction pattern is made using CuK_{α1} radiation.

Even more specifically, said X-ray powder diffraction pattern, made using CuK_{α1} radiation comprises peaks at degrees 2Θ (±0.1 degrees 2Θ) as contained in Table 3.

TABLE 3
X-ray powder diffraction pattern of the crystalline complex of FORM
1002 (only peaks up to 30° in 2 Θ are listed):

| | 2 Θ | Intensity I/I₀ |
|---|---|---|
| Peak No | [°] | [%] |
| 1 | 5.47 | 37.5 |
| 2 | 5.75 | 7.9 |
| 3 | 7.47 | 56.5 |
| 4 | 9.67 | 7.9 |
| 5 | 10.20 | 5.6 |
| 6 | 10.57 | 43.3 |
| 7 | 10.91 | 13.5 |
| 8 | 11.59 | 18.9 |
| 9 | 13.10 | 6.3 |
| 10 | 14.61 | 4.7 |
| 11 | 15.59 | 14.9 |
| 12 | 16.28 | 100.0 |
| 13 | 17.20 | 17.7 |
| 14 | 17.40 | 18.4 |
| 15 | 18.29 | 6.3 |
| 16 | 18.87 | 61.4 |
| 17 | 19.37 | 16.1 |
| 18 | 19.73 | 11.9 |
| 19 | 19.86 | 11.3 |
| 20 | 20.51 | 39.4 |
| 21 | 20.65 | 40.8 |
| 22 | 21.24 | 20.3 |
| 23 | 21.63 | 23.3 |
| 24 | 21.96 | 25.1 |
| 25 | 22.53 | 37.1 |
| 26 | 23.17 | 21.9 |
| 27 | 24.25 | 15.7 |
| 28 | 24.56 | 6.2 |
| 29 | 24.89 | 16.3 |
| 30 | 25.84 | 65.3 |

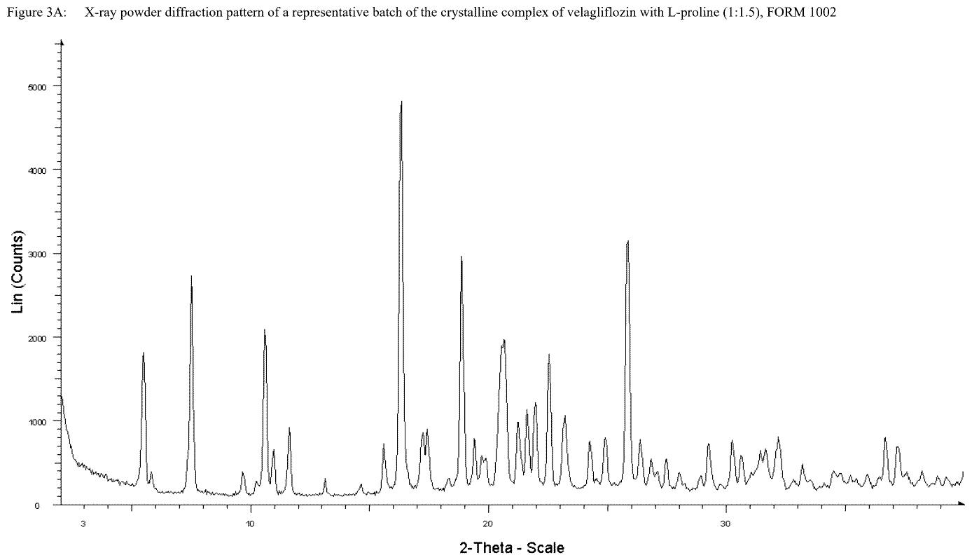

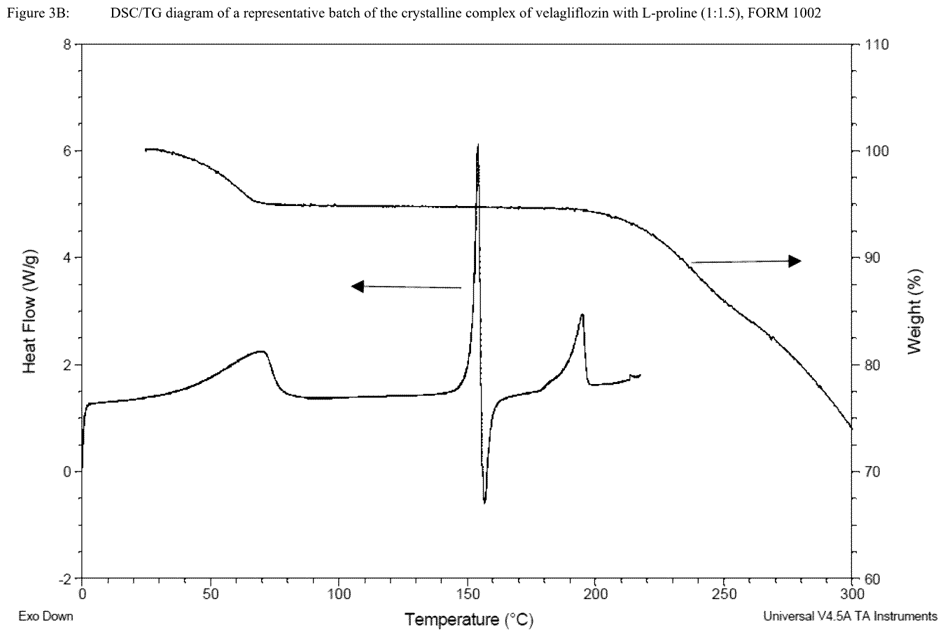

Even more specifically, said X-ray powder diffraction pattern, made using CuK_{α1} radiation comprises peaks at degrees 2Θ (±0.1 degrees 2Θ) as shown in FIG. 3A.

Said crystalline complex of velagliflozin with L-proline shows a weight loss by thermal gravimetry (TG). The observed weight loss indicates that the crystalline form contains water which may be bound by adsorption and/or may be part of the crystalline lattice, i.e the crystalline form may be present as a crystalline hydrate.

Furthermore, said crystalline complex of velagliflozin with L-proline is characterized by a dehydration noted around 50° C. (determined via DSC; evaluated as onset-temperature; heating rate 10 K/min). The obtained DSC curve is shown in FIG. 3B.

In another alternative embodiment, the crystalline complex has a content of water of about 1.5 mol per mol of velagliflozin, characterized in that the crystalline complex is a complex (1:1) between velagliflozin and L-proline; and characterized by an X-ray powder diffraction pattern that comprises peaks at 5.49, 14.45, 18.20 and 23.76 degrees 2Θ (±0.1 degrees 2Θ), wherein said X-ray powder diffraction pattern is made using CuK_{α1} radiation.

It is preferred, when said X-ray powder diffraction pattern further comprises peaks at 16.49, 17.29, 22.30 and 23.49 degrees 2Θ (±0.1 degrees 2Θ), wherein said X-ray powder diffraction pattern is made using CuK_{α1} radiation.

In particular, said X-ray powder diffraction pattern further comprises peaks at 11.52, 18.65, 19.43, 19.99, 20.33, 21.35, 23.11 and 26.34 degrees 2Θ (±0.1 degrees 2Θ), wherein said X-ray powder diffraction pattern is made using CuK_{α1} radiation.

More specifically, said X-ray powder diffraction pattern further comprises peaks at 6.36, 15.43, 19.16, 20.64, 20.99, 22.73, 24.34 and 26.04 degrees 2Θ (±0.1 degrees 2Θ), wherein said X-ray powder diffraction pattern is made using CuK_{α1} radiation.

Even more specifically, said X-ray powder diffraction pattern, made using CuK_{α1} radiation comprises peaks at degrees 2Θ (±0.1 degrees 2Θ) as contained in Table 4.

TABLE 4
X-ray powder diffraction pattern of the crystalline complex of FORM
1003 (only peaks up to 30° in 2 Θ are listed):

| | 2 Θ | Intensity I/I₀ |
|---|---|---|
| Peak No | [°] | [%] |
| 1 | 5.49 | 100.0 |
| 2 | 6.36 | 6.7 |
| 3 | 10.39 | 4.4 |
| 4 | 10.98 | 2.3 |
| 5 | 11.52 | 7.0 |
| 6 | 12.73 | 3.0 |
| 7 | 14.15 | 4.4 |
| 8 | 14.45 | 18.8 |
| 9 | 15.43 | 5.4 |
| 10 | 16.49 | 16.7 |
| 11 | 17.29 | 11.6 |
| 12 | 18.20 | 18.2 |
| 13 | 18.65 | 10.0 |
| 14 | 19.16 | 6.6 |
| 15 | 19.43 | 7.5 |
| 16 | 19.99 | 10.5 |
| 17 | 20.33 | 8.0 |
| 18 | 20.64 | 6.1 |
| 19 | 20.99 | 4.8 |
| 20 | 21.35 | 10.0 |
| 21 | 22.30 | 12.3 |
| 22 | 22.73 | 4.9 |
| 23 | 23.11 | 9.0 |
| 24 | 23.49 | 13.8 |
| 25 | 23.76 | 20.9 |
| 26 | 24.34 | 5.7 |
| 27 | 25.39 | 4.4 |
| 28 | 26.04 | 5.6 |
| 29 | 26.34 | 7.2 |
| 30 | 27.13 | 11.5 |

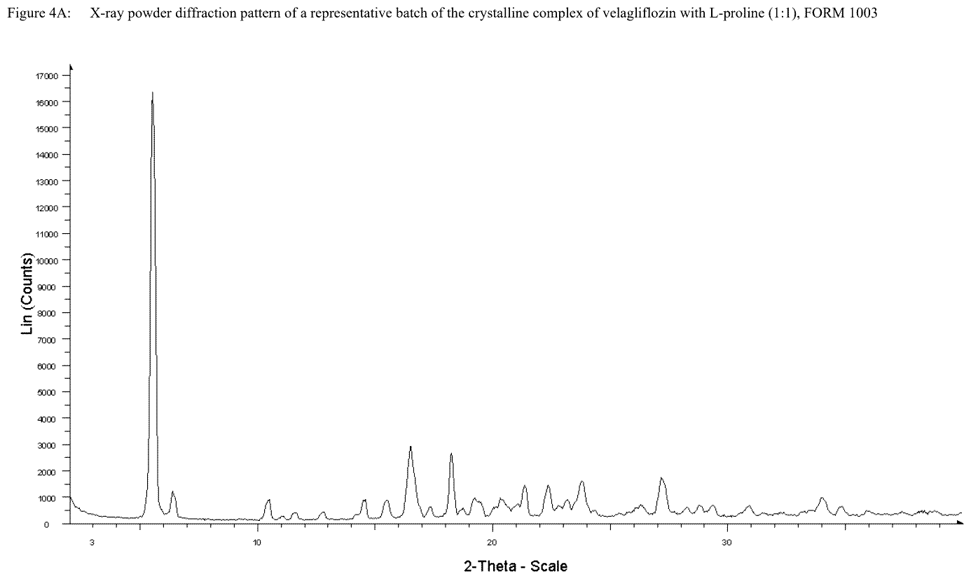

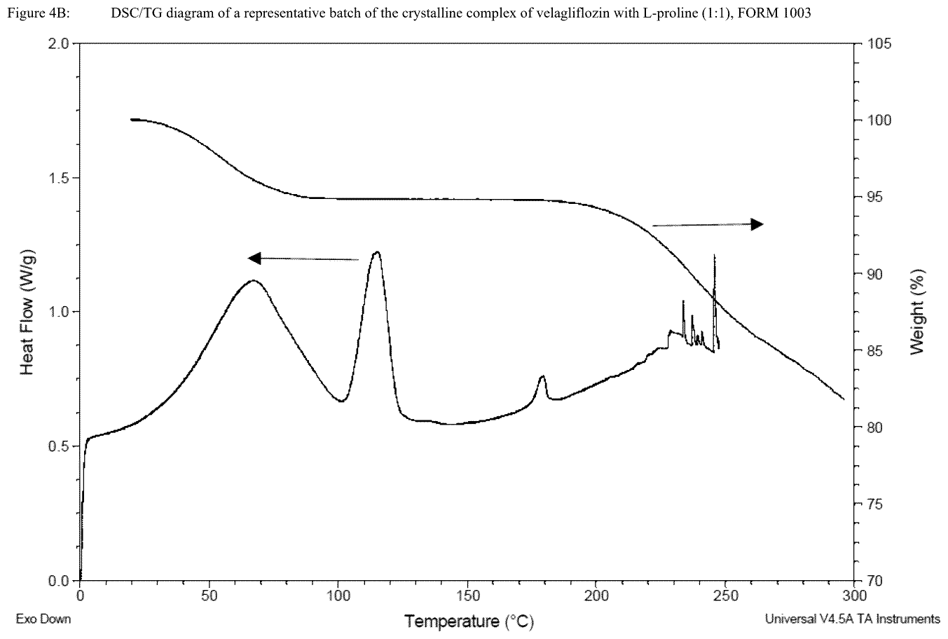

Even more specifically, said X-ray powder diffraction pattern, made using CuK_{α1} radiation comprises peaks at degrees 2Θ (±0.1 degrees 2Θ) as shown in FIG. 4A.

Said crystalline complex of velagliflozin with L-proline shows a weight loss by thermal gravimetry (TG). The observed weight loss indicates that the crystalline form contains water which may be bound by adsorption and/or may be part of the crystalline lattice, i.e the crystalline form may be present as a crystalline hydrate.

Furthermore, said crystalline complex of velagliflozin with L-proline is characterized by a dehydration noted around 50° C. (determined via DSC; evaluated as onset-temperature; heating rate 10 K/min). The obtained DSC curve is shown in FIG. 4B.

In another alternative embodiment, the crystalline complex has a content of water of about 2 mol per mol of velagliflozin, characterized in that the crystalline complex is a complex (1:2) between velagliflozin and L-proline; and characterized by an X-ray powder diffraction pattern that comprises peaks at 5.94, 16.40, 17.95 and 20.28 degrees 2Θ (±0.1 degrees 2Θ), wherein said X-ray powder diffraction pattern is made using Cuk 1 radiation.

It is preferred, when said X-ray powder diffraction pattern further comprises peaks at 18.49, 19.40, 19.72 and 23.17 degrees 2Θ (±0.1 degrees 2Θ), wherein said X-ray powder diffraction pattern is made using CuK_{α1} radiation.

In particular, said X-ray powder diffraction pattern further comprises peaks at 16.12, 17.56, 20.59, 22.21, 22.41, 23.67, 26.59 and 27.38 degrees 2Θ (±0.1 degrees 2Θ), wherein said X-ray powder diffraction pattern is made using CuK_{α1} radiation.

More specifically, said X-ray powder diffraction pattern further comprises peaks at 8.77, 15.24, 19.09, 21.50, 22.82, 24.10, 24.83 and 25.26 degrees 2Θ (±0.1 degrees 2Θ), wherein said X-ray powder diffraction pattern is made using CuK_{α1} radiation.

Even more specifically, said X-ray powder diffraction pattern, made using CuK_{α1} radiation comprises peaks at degrees 2Θ (±0.1 degrees 2Θ) as contained in Table 5.

TABLE 5
X-ray powder diffraction pattern of the crystalline complex of FORM
1004 (only peaks up to 30° in 2 Θ are listed):

| | 2 Θ | Intensity I/I₀ |
|---|---|---|
| Peak No | [°] | [%] |
| 1 | 5.94 | 100.0 |
| 2 | 8.77 | 18.0 |
| 3 | 8.97 | 17.9 |
| 4 | 9.60 | 7.0 |
| 5 | 11.94 | 10.9 |
| 6 | 13.25 | 8.3 |
| 7 | 13.38 | 9.4 |
| 8 | 15.24 | 20.9 |
| 9 | 16.12 | 30.0 |
| 10 | 16.40 | 50.2 |
| 11 | 17.56 | 37.6 |
| 12 | 17.95 | 44.2 |
| 13 | 18.49 | 43.6 |
| 14 | 19.09 | 19.3 |
| 15 | 19.40 | 42.9 |
| 16 | 19.72 | 30.2 |
| 17 | 20.28 | 78.3 |
| 18 | 20.59 | 23.5 |
| 19 | 21.50 | 21.1 |
| 20 | 21.78 | 15.1 |
| 21 | 22.21 | 28.6 |
| 22 | 22.41 | 24.4 |
| 23 | 22.82 | 18.7 |
| 24 | 23.17 | 43.1 |
| 25 | 23.67 | 39.9 |
| 26 | 24.10 | 19.4 |
| 27 | 24.83 | 19.4 |
| 28 | 25.26 | 19.5 |
| 29 | 26.59 | 25.2 |
| 30 | 27.38 | 26.4 |

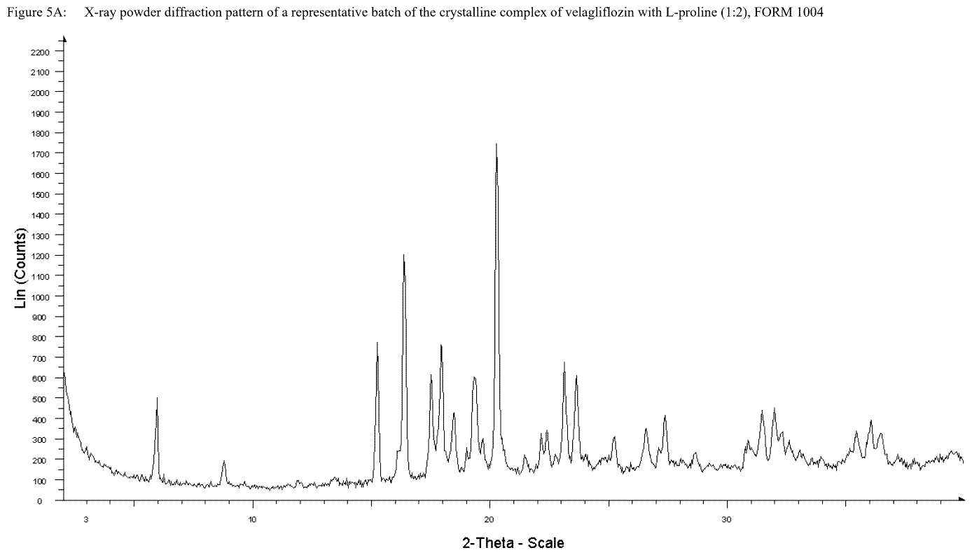

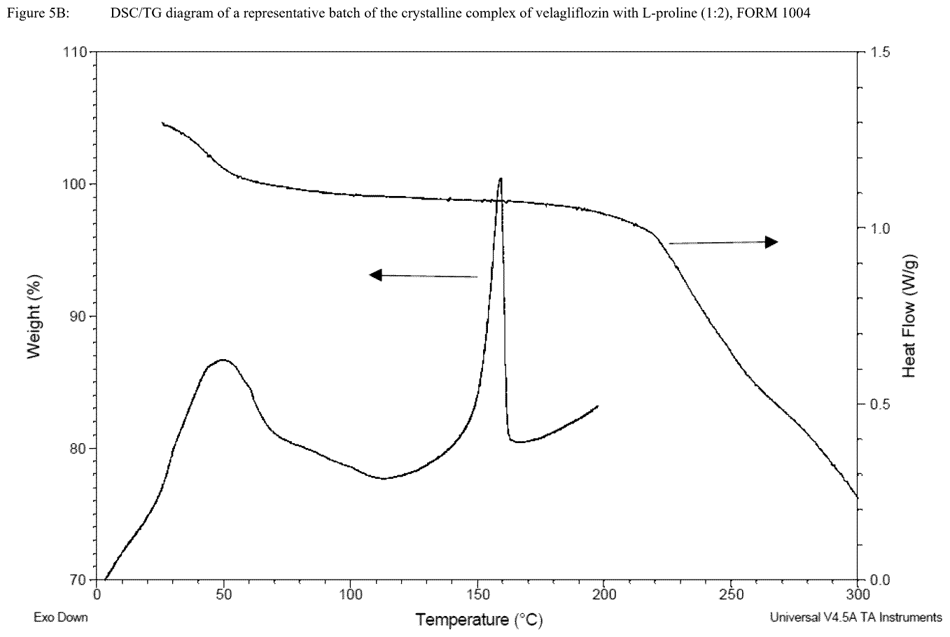

Even more specifically, said X-ray powder diffraction pattern, made using CuK_{α1} radiation comprises peaks at degrees 2Θ (±0.1 degrees 2Θ) as shown in FIG. 5A.

Said crystalline complex of velagliflozin with L-proline shows a weight loss by thermal gravimetry (TG). The observed weight loss indicates that the crystalline form contains water which may be bound by adsorption and/or may be part of the crystalline lattice, i.e. the crystalline form may be present as a crystalline hydrate.

Furthermore, said crystalline complex of velagliflozin with L-proline is characterized by a dehydration noted around 50° C. (determined via DSC; evaluated as onset-temperature; heating rate 10 K/min). The obtained DSC curve is shown in FIG. 5B.

In another alternative embodiment, the crystalline complex has a content of water of about 0 mol, characterized in that the crystalline complex is a complex (1:1) between velagliflozin and L-proline; and characterized by an X-ray powder diffraction pattern that comprises peaks at 5.66, 10.81, 17.03 and 17.75 degrees 2Θ (±0.1 degrees 2Θ), wherein said X-ray powder diffraction pattern is made using CuK_{α1} radiation.

It is preferred, when said X-ray powder diffraction pattern further comprises peaks at 6.48, 21.84, 22.00 and 23.82 degrees 2Θ (±0.1 degrees 2Θ), wherein said X-ray powder diffraction pattern is made using CuK_{α1} radiation.

In particular, said X-ray powder diffraction pattern further comprises peaks at 11.36, 14.41, 20.12, 21.67, 23.02, 23.10, 27.09 and 28.03 degrees 2Θ (±0.1 degrees 2Θ)), wherein said X-ray powder diffraction pattern is made using CuK_{α1} radiation.

More specifically, said X-ray powder diffraction pattern further comprises peaks at 16.10, 19.02, 19.83, 25.60, 25.85, 26.00, 27.34 and 28.47 degrees 2Θ (±0.1 degrees 2Θ), wherein said X-ray powder diffraction pattern is made using CuK_{α1} radiation.

Even more specifically, said X-ray powder diffraction pattern, made using CuK_{α1} radiation comprises peaks at degrees 2Θ (±0.1 degrees 2Θ) as contained in Table 6.

TABLE 6
X-ray powder diffraction pattern of the crystalline complex of FORM
1005 (only peaks up to 30° in 2 Θ are listed):

| | 2 Θ | Intensity I/I₀ |
|---|---|---|
| Peak No | [°] | [%] |
| 1 | 5.66 | 100.0 |
| 2 | 6.48 | 15.0 |
| 3 | 9.29 | 2.5 |
| 4 | 10.81 | 32.9 |
| 5 | 11.36 | 11.0 |
| 6 | 14.41 | 14.3 |
| 7 | 16.10 | 9.2 |
| 8 | 17.03 | 62.6 |
| 9 | 17.75 | 24.1 |
| 10 | 18.74 | 3.9 |
| 11 | 19.02 | 7.7 |
| 12 | 19.83 | 10.2 |
| 13 | 20.12 | 14.0 |
| 14 | 21.67 | 12.4 |
| 15 | 21.84 | 15.7 |
| 16 | 22.00 | 15.1 |
| 17 | 23.02 | 11.2 |
| 18 | 23.10 | 11.9 |
| 19 | 23.82 | 21.0 |
| 20 | 25.12 | 4.7 |
| 21 | 25.60 | 6.5 |
| 22 | 25.85 | 5.9 |
| 23 | 26.00 | 5.7 |
| 24 | 27.09 | 12.5 |
| 25 | 27.34 | 9.0 |
| 26 | 28.03 | 11.1 |
| 27 | 28.47 | 7.1 |
| 28 | 28.86 | 9.8 |

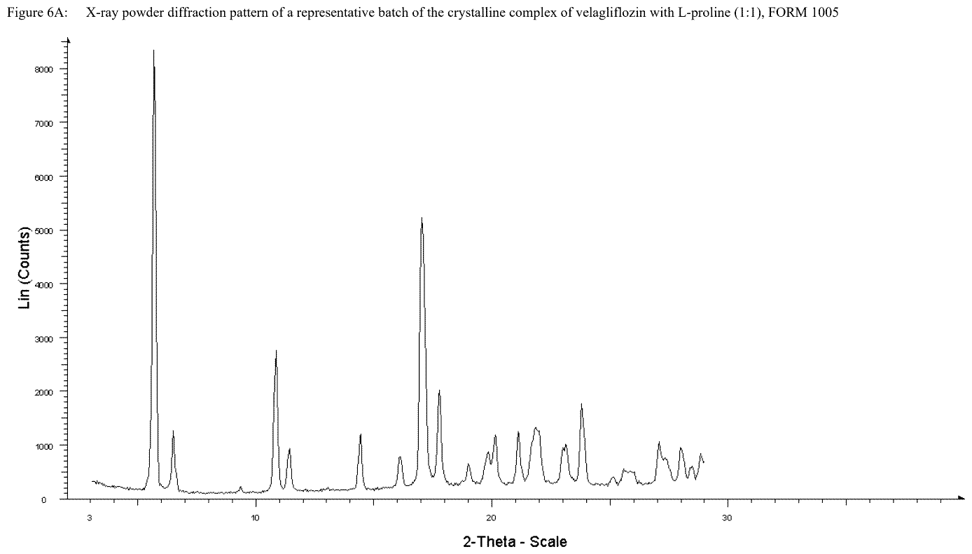

Even more specifically, said X-ray powder diffraction pattern, made using CuK_{α1} radiation comprises peaks at degrees 2Θ (±0.1 degrees 2Θ) as shown in FIG. 6A.

In another alternative embodiment, the crystalline complex has a content of water of about 0 mol, characterized in that the crystalline complex is a complex of velagliflozin and L-proline with a molar ratio in the range from about 1:1.4 to about 1:1.8; and characterized by an X-ray powder diffraction pattern that comprises peaks at 6.02, 16.64, 18.24 and 20.74 degrees 2Θ (±0.1 degrees 2Θ), wherein said X-ray powder diffraction pattern is made using CuK_{α1} radiation.

It is preferred, when said X-ray powder diffraction pattern further comprises peaks at 9.06, 15.49, 18.51 and 19.44 degrees 2Θ (±0.1 degrees 2Θ), wherein said X-ray powder diffraction pattern is made using CuK_{α1} radiation.

In particular, said X-ray powder diffraction pattern further comprises peaks at 17.79, 19.74, 22.70, 23.62, 24.06, 25.46, 27.03 and 27.88 degrees 2Θ (±0.1 degrees 2Θ), wherein said X-ray powder diffraction pattern is made using CuK_{α1} radiation.

More specifically, said X-ray powder diffraction pattern further comprises peaks at 12.06, 13.34, 20.21, 22.16, 23.04, 23.25, 24.48 and 27.31 degrees 2Θ (±0.1 degrees 2Θ), wherein said X-ray powder diffraction pattern is made using Cuk 1 radiation.

Even more specifically, said X-ray powder diffraction pattern, made using CuK_{α1} radiation comprises peaks at degrees 2Θ (±0.1 degrees 2Θ) as contained in Table 7.

TABLE 7
X-ray powder diffraction pattern of the crystalline complex of FORM
1006 (only peaks up to 30° in 2 Θ are listed):

| | 2 Θ | Intensity I/I₀ |
|---|---|---|
| Peak No | [°] | [%] |
| 1 | 5.03 | 3.4 |
| 2 | 6.02 | 100.0 |
| 3 | 9.06 | 23.5 |
| 4 | 9.68 | 4.8 |
| 5 | 12.06 | 7.6 |
| 6 | 13.34 | 6.6 |
| 7 | 15.49 | 34.4 |
| 8 | 16.64 | 59.8 |
| 9 | 17.79 | 17.7 |
| 10 | 18.24 | 33.9 |
| 11 | 18.51 | 25.8 |
| 12 | 19.44 | 26.3 |
| 13 | 19.74 | 10.2 |
| 14 | 20.21 | 6.5 |
| 15 | 20.74 | 63.7 |
| 16 | 22.16 | 7.7 |
| 17 | 22.70 | 12.2 |
| 18 | 23.04 | 7.3 |
| 19 | 23.25 | 7.1 |
| 20 | 23.62 | 11.5 |
| 21 | 24.06 | 22.2 |
| 22 | 24.48 | 7.0 |
| 23 | 25.46 | 9.5 |
| 24 | 26.15 | 5.1 |
| 25 | 27.03 | 9.4 |
| 26 | 27.31 | 6.0 |
| 27 | 27.88 | 12.6 |
| 28 | 28.51 | 5.0 |
| 29 | 28.84 | 5.3 |
| 30 | 29.56 | 4.0 |

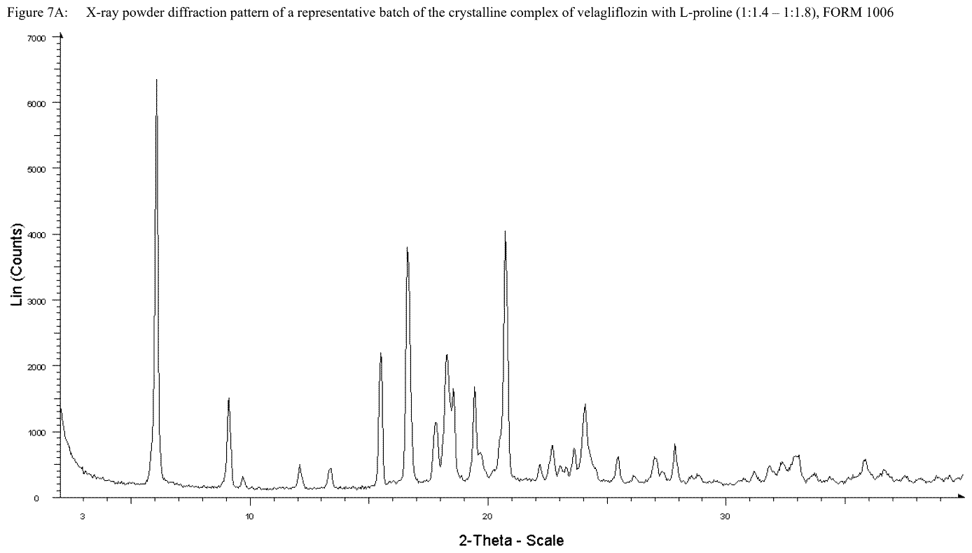

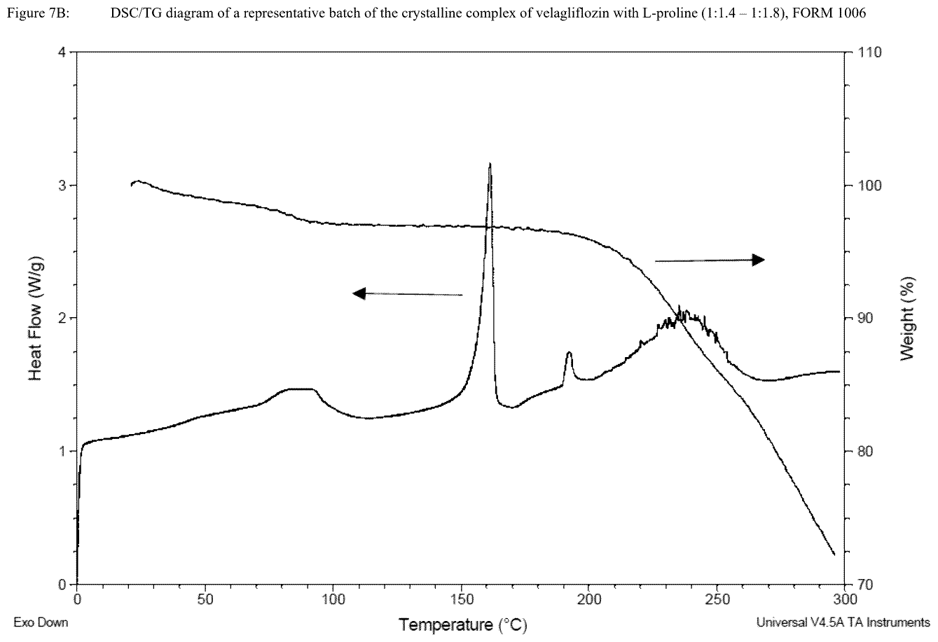

Even more specifically, said X-ray powder diffraction pattern, made using CuK_{α1} radiation comprises peaks at degrees 2Θ (±0.1 degrees 2Θ) as shown in FIG. 7A.

Furthermore, said crystalline complex of velagliflozin with L-proline is characterized by a melting point of 160° C. (determined via DSC; evaluated as onset-temperature; heating rate 10 K/min). The obtained DSC curve is shown in FIG. 7B.

In another alternative embodiment, the crystalline complex is in a hydrate form and is a complex (1:1) between velagliflozin and L-proline; and characterized by an X-ray powder diffraction pattern that comprises peaks at 8.69, 16.96, 18.12 and 24.02 degrees 2Θ (±0.1 degrees 2Θ), wherein said X-ray powder diffraction pattern is made using CuK_{α1} radiation.

It is preferred, when said X-ray powder diffraction pattern further comprises peaks at 10.78, 17.24, 20.48 and 21.39 degrees 2Θ (±0.1 degrees 2Θ), wherein said X-ray powder diffraction pattern is made using Cuk 1 radiation.

In particular, said X-ray powder diffraction pattern further comprises peaks at 6.43, 19.42, 20.06, 21.72, 22.39, 23.43, 27.85 and 28.92 degrees 2Θ (±0.1 degrees 2Θ), wherein said X-ray powder diffraction pattern is made using CuK_{α1} radiation.

More specifically, said X-ray powder diffraction pattern further comprises peaks at 11.45, 14.58, 16.09, 23.12, 26.33, 27.07, 27.36 and 29.46 degrees 2□ (±0.1 degrees 2Θ), wherein said X-ray powder diffraction pattern is made using CuK_{α1} radiation.

Even more specifically, said X-ray powder diffraction pattern, made using CuK_{α1} radiation comprises peaks at degrees 2Θ (±0.1 degrees 2Θ)) as contained in Table 8.

TABLE 8
X-ray powder diffraction pattern of the crystalline complex of FORM
1007 (only peaks up to 30° in 2 Θ are listed):

| | 2 Θ | Intensity I/I₀ |
|---|---|---|
| Peak No | [°] | [%] |
| 1 | 5.69 | 100.0 |
| 2 | 6.43 | 8.5 |
| 3 | 10.78 | 8.6 |
| 4 | 11.45 | 3.9 |
| 5 | 12.99 | 1.6 |
| 6 | 14.58 | 5.9 |
| 7 | 15.19 | 1.4 |
| 8 | 16.09 | 3.8 |
| 9 | 16.43 | 1.7 |
| 10 | 16.96 | 18.9 |
| 11 | 17.24 | 11.8 |
| 12 | 18.12 | 24.2 |
| 13 | 18.94 | 2.8 |
| 14 | 19.42 | 6.2 |
| 15 | 20.06 | 6.5 |
| 16 | 20.48 | 9.5 |
| 17 | 21.37 | 10.8 |
| 18 | 21.72 | 7.3 |
| 19 | 22.39 | 8.8 |
| 20 | 23.12 | 5.5 |
| 21 | 23.43 | 8.3 |
| 22 | 24.02 | 12.1 |
| 23 | 25.56 | 2.5 |
| 24 | 25.94 | 3.3 |
| 25 | 26.33 | 4.7 |
| 26 | 27.07 | 4.1 |
| 27 | 27.36 | 4.3 |
| 28 | 27.85 | 8.0 |
| 29 | 28.92 | 7.2 |
| 30 | 29.46 | 5.6 |

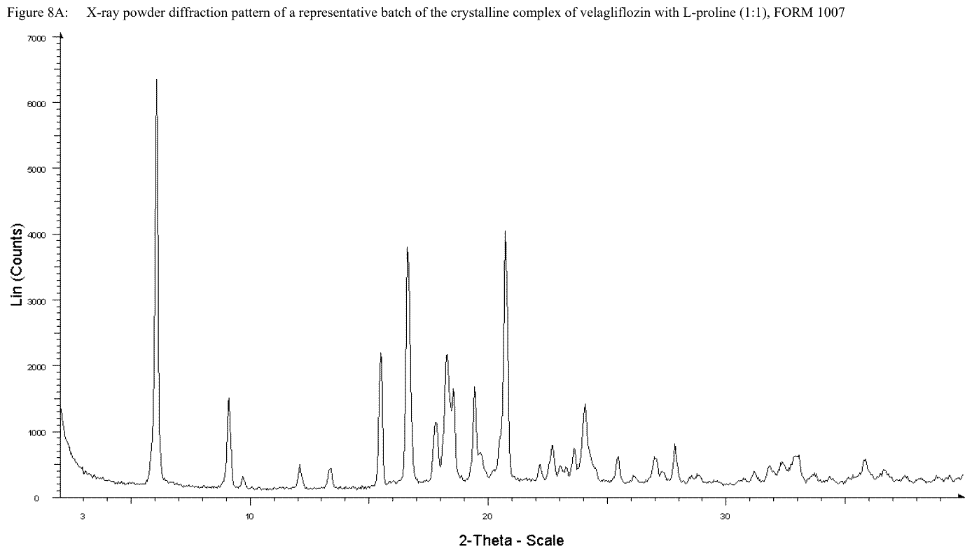

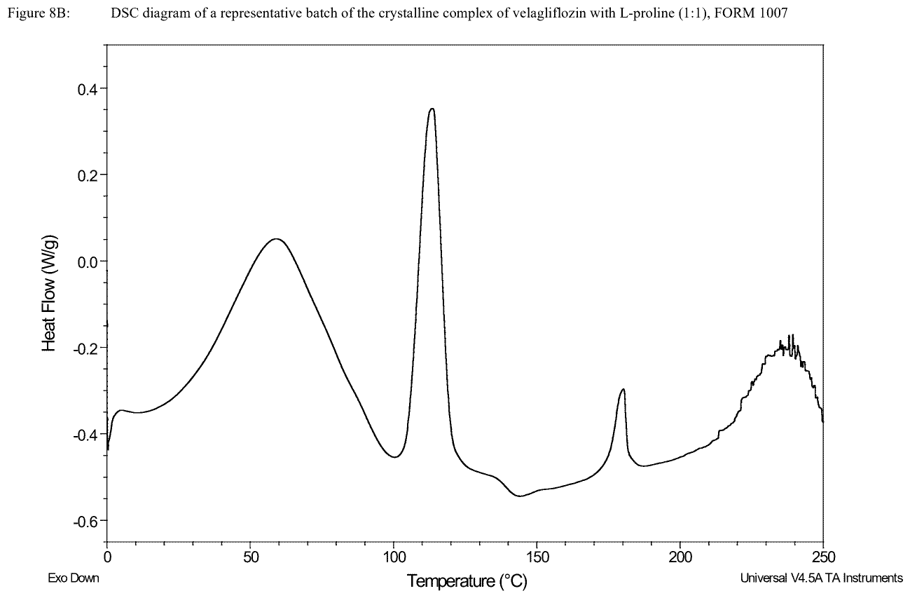

Even more specifically, said X-ray powder diffraction pattern, made using CuK_{α1} radiation comprises peaks at degrees 2Θ (±0.1 degrees 2Θ) as shown in FIG. 8A.

Furthermore, said crystalline complex of velagliflozin with L-proline is characterized by a dehydration noted around 60° C. (determined via DSC; evaluated as onset-temperature; heating rate 10 K/min). The obtained DSC curve is shown in FIG. 8B.

In another alternative embodiment, the crystalline complex has a content of tetrahydrofuran of about 0.4 mol per mol of velagliflozin, characterized in that the crystalline complex is a complex (1:1) between velagliflozin and L-proline; and characterized by an X-ray powder diffraction pattern that comprises peaks at 4.07, 17.21, 20.30 and 23.50 degrees 2Θ (±0.1 degrees 2Θ), wherein said X-ray powder diffraction pattern is made using CuK_{α1} radiation.

It is preferred, when said X-ray powder diffraction pattern further comprises peaks at 16.09, 18.49, 21.42 and 25.71 degrees 2Θ (±0.1 degrees 2Θ), wherein said X-ray powder diffraction pattern is made using CuK_{α1} radiation.

In particular, said X-ray powder diffraction pattern further comprises peaks at 14.74, 18.87, 21.05, 22.00, 23.71, 24.68, 24.86, and 25.24 degrees 2Θ (±0.1 degrees 2Θ), wherein said X-ray powder diffraction pattern is made using CuK_{α1} radiation.

More specifically, said X-ray powder diffraction pattern further comprises peaks at 4.43, 12.05, 15.30, 16.67, 19.46, 22.63, 26.29 and 26.42 degrees 2Θ (±0.1 degrees 2Θ), wherein said X-ray powder diffraction pattern is made using CuK_{α1} radiation.

Even more specifically, said crystalline complex is characterized by an X-ray powder diffraction pattern, made using CuK_{α1} radiation, which comprises peaks at degrees 2Θ (±0.1 degrees 2Θ) as contained in Table 9.

TABLE 9
X-ray powder diffraction pattern of the crystalline complex of FORM
1008 (only peaks up to 30° in 2 Θ are listed):

| | 2 Θ | Intensity I/I₀ |
|---|---|---|
| Peak No | [°] | [%] |
| 1 | 4.07 | 58.0 |
| 2 | 4.43 | 12.5 |
| 3 | 6.23 | 3.1 |
| 4 | 10.14 | 3.8 |
| 5 | 12.05 | 6.9 |
| 6 | 13.05 | 3.7 |
| 7 | 13.36 | 3.9 |
| 8 | 13.62 | 3.2 |
| 9 | 14.33 | 3.6 |
| 10 | 14.74 | 17.0 |
| 11 | 15.30 | 9.0 |
| 12 | 16.09 | 25.1 |
| 13 | 16.67 | 8.4 |
| 14 | 17.21 | 76.5 |
| 15 | 18.49 | 23.2 |
| 16 | 18.87 | 19.0 |
| 17 | 19.46 | 14.1 |
| 18 | 20.30 | 100.0 |
| 19 | 21.05 | 17.3 |
| 20 | 21.42 | 26.7 |
| 21 | 22.00 | 20.6 |
| 22 | 22.63 | 10.9 |
| 23 | 23.50 | 31.7 |
| 24 | 23.71 | 18.8 |
| 25 | 24.68 | 16.6 |
| 26 | 24.86 | 15.1 |
| 27 | 25.24 | 15.2 |
| 28 | 25.71 | 21.2 |
| 29 | 26.29 | 14.9 |
| 30 | 26.42 | 14.4 |

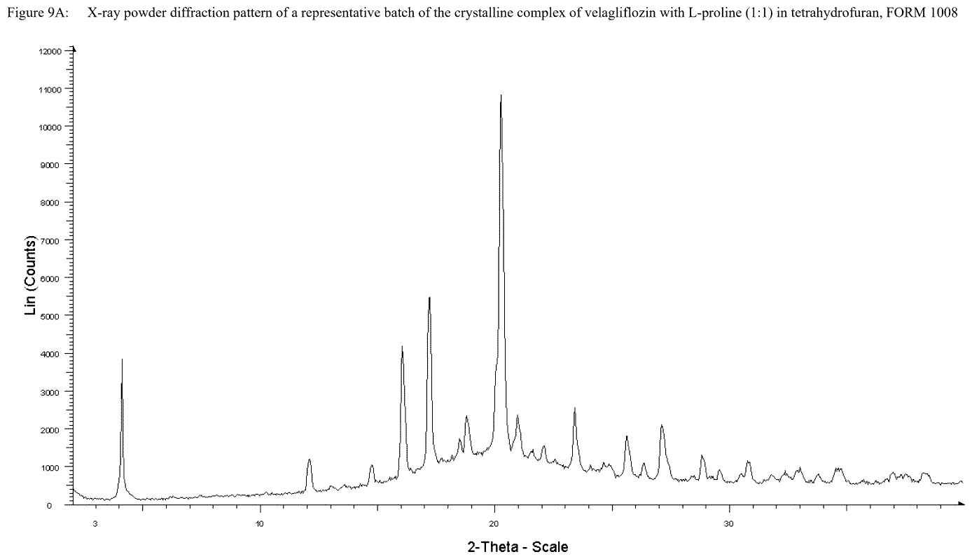

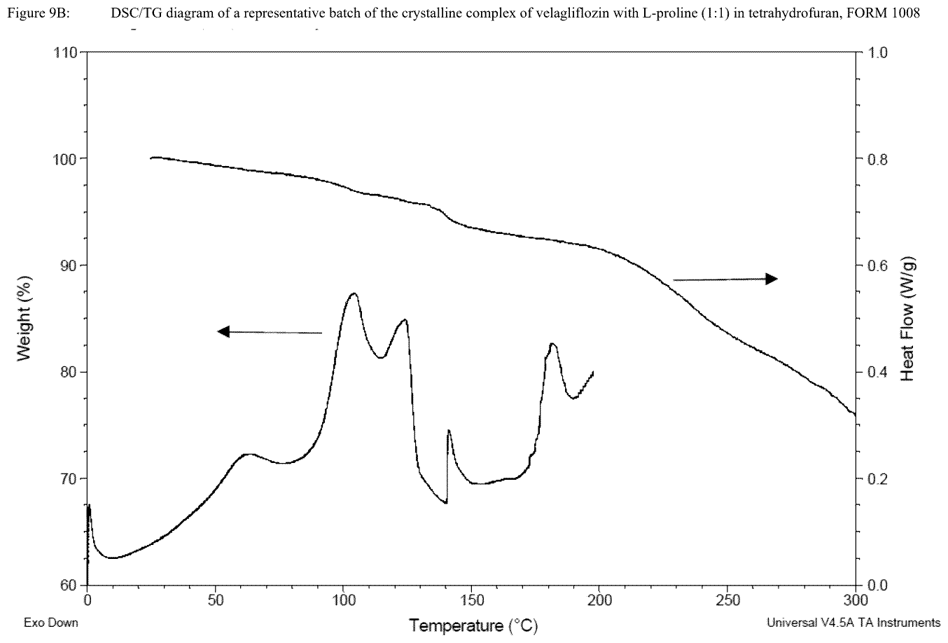

Even more specifically, said X-ray powder diffraction pattern, made using CuK_{α1} radiation comprises peaks at degrees 2Θ (±0.1 degrees 2Θ) as shown in FIG. 9A.

Furthermore, said crystalline complex of velagliflozin with L-proline is characterized by a complex DSC profile with a dehydration/desolvation noted around 60° C. (determined via DSC; evaluated as onset-temperature; heating rate 10 K/min). The obtained DSC curve is shown in FIG. 9B.

In another preferred embodiment the crystalline complex is a complex between velagliflozin and theobromine and it has a content of water of about 0 mol, i.e. it is a water-free/anhydrous co-crystal of velagliflozin with theobromine.

In yet another preferred embodiment, the crystalline complex is a complex (1:1) between velagliflozin and theobromine; and characterized by an X-ray powder diffraction pattern that comprises peaks at 18.22, 20.15, 22.56 and 25.36 degrees 2Θ (±0.1 degrees 2Θ), wherein said X-ray powder diffraction pattern is made using CuK_{α1} radiation.

It is preferred, when said X-ray powder diffraction pattern further comprises peaks at 14.19, 27.01, 34.11 and 38.63 degrees 2Θ (±0.1 degrees 2Θ), wherein said X-ray powder diffraction pattern is made using Cuk 1 radiation.

In particular, said X-ray powder diffraction pattern further comprises peaks at 30.65, 30.95, 32.27, and 36.89 degrees 2Θ (±0.1 degrees 2Θ), wherein said X-ray powder diffraction pattern is made using Cuk 1 radiation.

More specifically, said X-ray powder diffraction pattern further comprises peaks at 15.23, 32.08, 36.06, 37.54, and 39.82 degrees 2Θ (±0.1 degrees 2Θ), wherein said X-ray powder diffraction pattern is made using CuK_{α1} radiation.

Even more specifically, said crystalline complex is characterized by an X-ray powder diffraction pattern, made using CuK_{α1} radiation, which comprises peaks at degrees 2Θ (±0.1 degrees 2Θ) as contained in Table 10.

TABLE 10
X-ray powder diffraction pattern of
the crystalline complex of FORM 1009:

| | 2 Θ | Intensity I/I₀ |
|---|---|---|
| Peak No | [°] | [%] |
| 1 | 14.19 | 10.0 |
| 2 | 15.23 | 4.9 |
| 3 | 18.22 | 23.9 |
| 4 | 20.15 | 100.0 |
| 5 | 22.56 | 22.5 |
| 6 | 25.36 | 11.0 |
| 7 | 27.01 | 8.6 |
| 8 | 30.65 | 8.0 |
| 9 | 30.95 | 6.0 |
| 10 | 32.08 | 4.5 |
| 11 | 32.27 | 5.2 |
| 12 | 34.11 | 10.5 |
| 13 | 36.06 | 4.7 |
| 14 | 36.89 | 5.2 |
| 15 | 37.54 | 5.2 |
| 16 | 38.61 | 9.8 |
| 17 | 39.82 | 5.2 |

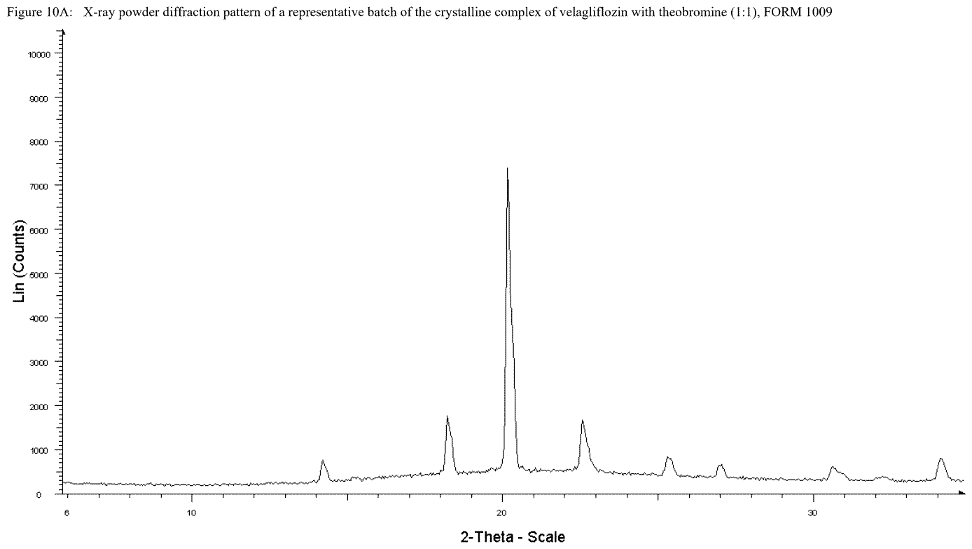

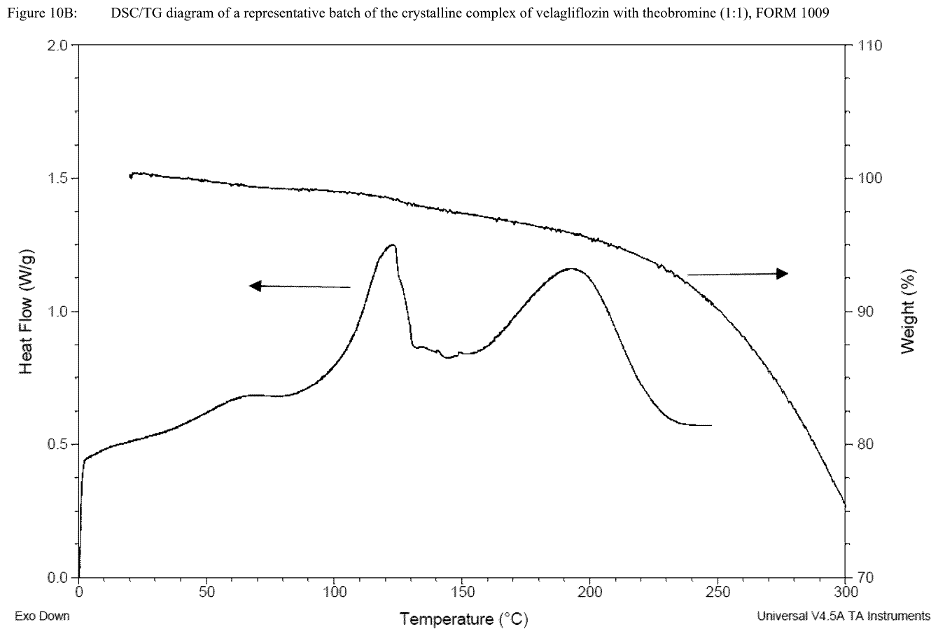

Even more specifically, said X-ray powder diffraction pattern, made using CuK_{α1} radiation comprises peaks at degrees 2Θ (±0.1 degrees 2Θ) as shown in FIG. 10A.

Furthermore, said crystalline complex of velagliflozin with theobromine is characterized by a melting point of 120° C. (determined via DSC; evaluated as onset-temperature; heating rate 10 K/min). The obtained DSC curve is shown in FIG. 10B.

The crystalline complexes according to the present invention have advantageous physicochemical properties which are beneficial in the preparation of a pharmaceutical composition. In particular, such crystalline complexes have a high physical and chemical stability under various environmental conditions and during the production of a medicament. For example, the crystals can be obtained in a shape and particle size which are particularly suitable in a production method for solid pharmaceutical formulations. In addition, the crystals show a high mechanical stability that allows grinding of the crystals. Furthermore, the crystalline complexes do not show a high tendency to absorb moisture and are chemically stable, i.e., the crystalline complexes allow the production of a solid pharmaceutical formulation with a long shelf life. On the other hand, the crystalline complexes have a favorably high solubility over a wide pH-range which is advantageous in solid pharmaceutical formulations for oral administration.

The X-ray powder diffraction patterns are recorded, within the scope of the present invention, using a BRUKER D8-Advance diffractometer in reflection mode fitted with a location-sensitive detector (OED) and a Cu-anode as X-ray source (CuK_{α1} radiation, λ=1.54056 Å, 35 kV, 40 mA). In the tables the values “2Θ [°]” denote the angle of diffraction in degrees and the values “d[Å]” denote the specified distances in Å between the lattice planes. The intensity shown in the figures is given in units of cps (counts per second).

In order to allow for experimental error, the above described 2 Θ values should be considered accurate to ±0.1 degrees 2 Θ, in particular ±0.05 degrees 2 Θ. That is to say, when assessing whether a given sample of crystals of velagliflozin is the crystalline form in accordance with the invention, a 2 Θ value which is experimentally observed for the sample should be considered identical with a characteristic value described above if it falls within ±0.1 degrees 2 Θ of the characteristic value, in particular if it falls within ±0.05 degrees 2 Θ of the characteristic value.

The melting point is determined by DSC (Differential Scanning calorimetry) using a DSC Q200 (TA-Instrument/Waters). The weight loss is determined by thermal gravimetry (TG) using a TGA Q500 (TA-Instruments/Waters).

A further aspect of the present invention relates to a method for making the one or more crystalline complexes of the present invention as defined hereinbefore and hereinafter, said method comprising the following steps:
(a) preparing a solution of velagliflozin and a co-crystal forming agent in a solvent or a mixture of solvents;
(b) storing the solution to precipitate the crystalline complex out of solution;
(c) removing the precipitate from the solution; and
(d) drying the precipitate optionally until any excess of said solvent or mixture of solvents has been removed.

According to step (a) a solution of velagliflozin and the co-crystal forming agent in a solvent or a mixture of solvents is prepared. Preferably, the solution is saturated or at least nearly saturated or even supersaturated with respect to the respective crystalline complex. In step (a) velagliflozin may be dissolved in a solution comprising the co-crystal forming agent; or the co-crystal forming agent may be dissolved in a solution comprising velagliflozin. According to an alternative procedure velagliflozin is dissolved in a solvent or mixture of solvents to yield a first solution; and the co-crystal forming agent is dissolved in a solvent or mixture of solvents to yield a second solution. Thereafter, said first solution and said second solution are combined to form the solution according to step (a).

Preferably, the molar ratio of the co-crystal forming agent and velagliflozin in the solution corresponds to the molar ratio of the co-crystal forming agent and velagliflozin in the respective crystalline complex to be obtained.

Therefore, a preferred molar ratio is in the range from about 1:1 to about 1:3.

Suitable solvents are preferably selected from the group consisting of C₁₋₄-alkanols, water, ethylacetate, acetonitrile, acetone, diethylether, tetrahydrofuran, and mixture of two or more of these solvents.

More preferred solvents are selected from the group consisting of methanol, ethanol, isopropanol, water and mixture of two or more of these solvents, in particular mixtures of one or more of said organic solvents with water.

Particularly preferred solvents are selected from the group consisting of ethanol, isopropanol, water and mixtures of ethanol and/or isopropanol with water.

In case a mixture of water and one or more C₁₋₄-alkanols, in particular of methanol, ethanol and/or isopropanol, most preferably of ethanol, is taken, a preferred volume ratio of water:C₁₋₄-alkanol is in the range from about 99:1 to about 1:99; more preferably from about 50:1 to about 1:80; even more preferably from about 10:1 to about 1:60.

Preferably the step (a) is carried out at about room temperature (about 20° C.) or at an elevated temperature up to about the boiling point of the solvent or mixture of solvents used.

According to a preferred embodiment the starting material of velagliflozin and/or of the co-crystal forming agent and/or of the solvent and mixtures of solvents contain an amount of H₂O, which is at least the quantity required to form a hydrate of velagliflozin; in particular at least 1 mol, preferably at least 1.5 mol of water per mol of velagliflozin. Even more preferably the amount of water is at least 2 mol of water per mol of velagliflozin. This means that either velagliflozin as starting material or the co-crystal forming agent or said solvent or mixture of solvents, or said compounds and/or solvents in combination contain an amount of H₂O as specified above. For example if the starting material of velagliflozin or of the co-crystal forming agent in step (a) does contain sufficient water as specified above, a water content of the solvent(s) is not mandatory.

In order to reduce the solubility of the one or more crystalline complexes according to this invention in the solution, in step (a) and/or in step (b) one or more antisolvents may be added, preferably during step (a) or at the beginning of step (b). Water is an example of a suitable antisolvent. The amount of antisolvent is preferably chosen to obtain a supersaturated or saturated solution with respect to the respective crystalline complex.

In step (b) the solution is stored for a time sufficient to obtain a precipitate, i.e. the respective crystalline complex. The temperature of the solution in step (b) is about the same as or lower than in step (a). During storage the temperature of the solution is preferably lowered, preferably to a temperature in the range of 20° C. to 0° C. or even lower. Step (b) can be carried out with or without stirring. As known to the one skilled in the art by the period of time and the difference of temperature in step (b) the size, shape and quality of the obtained crystals can be controlled. Furthermore, the crystallization may be induced by methods as known in the art, for example by mechanical means such as scratching or rubbing the contact surface of the reaction vessel for example with a glass rod. Optionally the (nearly) saturated or supersaturated solution may be inoculated with seed crystals.

In step (c) the solvent(s) can be removed from the precipitate by known methods as for example filtration, suction filtration, decantation or centrifugation.

In step (d) an excess of the solvent(s) is removed from the precipitate by methods known to the one skilled in the art as for example by reducing the partial pressure of the solvent(s), preferably in vacuum, and/or by heating above ca. 20° C., preferably in a temperature range below 100° C., even more preferably below 85° C.

Velagliflozin may be synthesized by methods as specifically and/or generally described or cited in the international applications WO 2007/093610 and WO 2007/128749. Furthermore, the biological properties of velagliflozin may be investigated as it is described in the international applications WO 2007/093610 and WO 2007/128749.

The one or more crystalline complexes in accordance with the invention are preferably employed as drug active substance in substantially pure form, that is to say, essentially free of other crystalline forms of velagliflozin. Nevertheless, the invention also embraces the crystalline complex in accordance with the invention in admixture with another crystalline form or forms of velagliflozin. Should the drug active substance be a mixture of crystalline forms, it is preferred that the substance comprises at least 50%-weight, even more preferably at least 90%-weight, most preferably at least 95%-weight of the crystalline complex as described herein.

In view of their ability to inhibit the SGLT activity, the one or more crystalline complexes as defined hereinbefore or hereinafter and/or the pharmaceutical composition as defined hereinbefore or hereinafter and/or the medicament as defined hereinbefore or hereinafter are suitable for the treatment and/or preventive treatment of conditions or diseases which may be affected by the inhibition of the SGLT activity, particularly the SGLT-2 activity. The one or more crystalline complexes as defined hereinbefore or hereinafter are also suitable for the preparation of pharmaceutical compositions for the treatment and/or preventive treatment of conditions or diseases which may be affected by the inhibition of the SGLT activity, particularly the SGLT-2 activity. Therefore, the one or more crystalline complexes according to the invention as defined hereinbefore or hereinafter are suitable for the treatment of diabetes, in particular feline diabetes and/or canine diabetes.

The dosage required to achieve the corresponding activity for treatment and/or prevention usually depends on the patient, the nature and gravity of the illness or condition and the method and frequency of administration and is for the patient's doctor/veterinarian to decide. Expediently, the dosage may be from 1 to 100 mg by intravenous route, and 1 to 1000 mg by oral route, in each case administered 1 to 4 times a day. For this purpose, the pharmaceutical compositions according to this invention preferably comprise the one or more crystalline complexes according to the invention together with one or more inert conventional carriers and/or diluents. Such pharmaceutical compositions may be formulated as conventional galenic preparations, such as plain or coated tablets, capsules, powders, suspensions or suppositories.

### Examples

The following examples serve to further illustrate the present invention; but the same should not be construed as a limitation of the scope of the invention disclosed herein.

In the foregoing and following text, H atoms of hydroxyl groups are not explicitly shown in every case in the structural formulae. The following example of synthesis serves to illustrate a method of preparing velagliflozin and its crystalline complex with different co-crystal forming agents. It is to be regarded only as a possible method described by way of example, without restricting the invention to its contents. The terms “room temperature” and “ambient temperature” are used interchangeably and denote temperatures of about 20° C.

### Preparation of Velagliflozin:

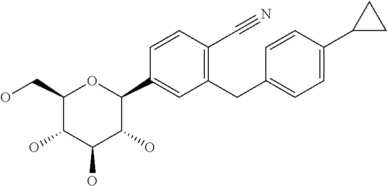 [SMILES: N#Cc1ccc([C@@H]2O[C@H](CO)[C@@H](O)[C@H](O)[C@H]2O)cc1Cc1ccc(C2CC2)cc1]

Velagliflozin is prepared as disclosed in WO2014/016381 on pages 13-18.

A crystalline complex (1:1) of velagliflozin with L-proline is prepared as disclosed in WO2014/016381 on page 18. The resulting crystalline complex is a monohydrate co-crystal and characterized by a molar ratio of velagliflozin and L-proline and crystal water of 1:1:1.

### Preparation of Form 1000:

Step 1: 10 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 1 ml of ethyl acetate and the resulting suspension is solubilized for 1 h at 60° C. The suspension is cooled by 0.01° C./min down to 5° C. The cooled suspension is filtered using a 0.2 μm filter and dried under vacuum at RT for 1 h.

Complementary NMR analysis confirms the chemical integrity of the velagliflozin material, the presence of 2.9 mole eq. of proline and the absence of residual solvent.

Several batches of the crystalline complex according to the above preparation are obtained. The X-ray powder diffraction patterns coincide. The melting points are determined via DSC and evaluated as onset-temperature.

DSC thermogram show a large and narrow endothermic peak at 195° C. associated with the melting transition of Form 1000. From these results it could be concluded that the crystalline complex was present in an anhydrous form.

The X-ray powder diffraction pattern as contained in Table 1 and as depicted in FIG. 1A and the DSC and TG diagram in FIG. 1B correspond to a batch with a melting point of approximately 195° C.

### Preparation of Form 1001:

Step 1: 10 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 1 ml of acetone and the resulting suspension is solubilized for 3 h at RT. After slow evaporation at RT a white solid was obtained.
Step 2 (alternative 2): Alternatively, the glassy material from step 1 is mixed with a mixture of acetone and ethanol (ratio 10:110) and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 3): The glassy material from step 1 is mixed with 100 μl of methyl-THF and the resulting suspension is solubilized for 1 h at 60° C. The suspension is cooled by 0.01° C./min down to 5° C. The cooled suspension is filtered using a 0.2 μm filter and dried under vacuum at RT for 1 h.

Complementary NMR analysis confirms the chemical integrity of the velagliflozin material, the presence of 1 mole eq. of proline and the presence of 0.6 mol acetone of residual solvent or in the absence of residual solvent.

Several batches of the crystalline complex according to the above preparation are obtained. The X-ray powder diffraction patterns coincide. The melting points are determined via DSC and evaluated as onset-temperature. DSC thermograms show no significant thermal event between 0° C. and 80° C. and a broad endothermic event around 80° C. associated with dehydration of crystalline material. From these results it could be concluded that the crystalline complex was present in a hydrated form.

The X-ray powder diffraction pattern as contained in Table 2 and as depicted in FIG. 2A and the DSC and TG diagram in FIG. 2B correspond to a batch with a dehydration noted at around 80° C.

### Preparation of Form 1002:

Step 1: 30 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 1 ml of dimethylcarbonate and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 2): Alternatively, the glassy material from step 1 is mixed with a mixture of water and 2-propanol (ratio 12:110) and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 3): The glassy material from step 1 is mixed 1 ml of acetonitrile and the resulting suspension is solubilized for 1 h at 60° C. The suspension is cooled by 0.01° C./min down to 5° C. The cooled suspension is filtered using a 0.2 μm filter and dried under vacuum at RT for 1 h.

### Alternative Preparations of Form 1002:

Step 1 (alternative 1): 40 mg of crystalline complex (1:1) of velagliflozin with L-proline is suspended in 200 μl of Acetonitrile. The suspension is stirred 30 days at RT and then filtered using a 0.2 μm filter.
Step 1 (alternative 2): 20 mg of crystalline complex (1:1) of velagliflozin with L-proline is suspended in 100 μl of a mixture of water and dioxane (ratio 0.03:97 volume). The suspension is stirred 30 days at RT and then filtered using a 0.2 μm filter.

Complementary NMR analysis confirms the chemical integrity of the velagliflozin material, the presence of 1.5 mole eq. of proline and the presence of trace of residual dioxane (<0.1 mol) or in the absence of residual solvent. Several batches of the crystalline complex according to the above preparation are obtained. The X-ray powder diffraction patterns coincide. The melting points are determined via DSC and evaluated as onset-temperature. DSC thermograms show a weak and broad endothermic event between RT and 90° C. associated with the dehydration of crystalline material. An endothermic peak at ~150° C. is associated with the melting transition of crystalline material obtained after dehydration. From these results it could be concluded that the crystalline complex was present in a hydrated form.

The X-ray powder diffraction pattern as contained in Table 3 and as depicted in FIG. 3A and the DSC and TG diagram in FIG. 3B correspond to a batch with a dehydration noted at around 50° C.

### Preparation of Form 1003:

Step 1: 10 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 100 μl of ethanol and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 2): Alternatively, the glassy material from step 1 is mixed with 1 ml of xylene and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 3): The glassy material from step 1 is mixed with 300 μl of methanol and the resulting suspension is solubilized for 1 h at 60° C. The suspension is cooled by 0.01° C./min down to 5° C. The cooled suspension is filtered using a 0.2 μm filter and dried under vacuum at RT for 1 h.

Complementary NMR analysis confirms the chemical integrity of the velagliflozin material, the presence of 1.0 mole eq. of proline and the presence of trace of residual dioxane (<0.1 mol) or in the absence of residual solvent. Several batches of the crystalline complex according to the above preparation are obtained. The X-ray powder diffraction patterns coincide. The melting points are determined via DSC and evaluated as onset-temperature. DSC thermograms show a weak and broad endothermic event between RT and 90° C. associated with the dehydration of crystalline material. From these results it could be concluded that the crystalline complex was present in a hydrated form.

The X-ray powder diffraction pattern as contained in Table 4 and as depicted in FIG. 4A and the DSC and TG diagram in FIG. 4B correspond to a batch with a dehydration noted at around 50° C.

### Preparation of Form 1004:

Step 1: 10 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 1 ml of anisole and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 2): Alternatively, the glassy material from step 1 is mixed with 1 ml of xylene and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 3): The glassy material from step 1 is mixed with 500 μl of cyclopropylmethylether and the resulting suspension is solubilized for 1 h at 60° C. The suspension is cooled by 0.01° C./min down to 5° C. The cooled suspension is filtered using a 0.2 μm filter and dried under vacuum at RT for 1 h.

The obtained solid is stored 7 days under room conditions.

Several batches of the crystalline complex according to the above preparation are obtained. The X-ray powder diffraction patterns coincide. The melting points are determined via DSC and evaluated as onset-temperature. From these results it could be concluded that the crystalline complex was present in a hydrated form.

The X-ray powder diffraction pattern as contained in Table 5 and as depicted in FIG. 5A and the DSC and TG diagram in FIG. 5B correspond to a batch with a dehydration noted at around 50° C.

### Preparation of Form 1005:

Step 1: Step 1: 10 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 400 μl of Water/Ethanol 2/20 (volume ratio) binary solvent and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 3: Solid material is heated to 95° C.

The X-ray powder diffraction patterns recorded at 95° C. is associated with the Form 1005.

Form 1005 being obtained from dehydration at 95° C. of Form 1003, we conclude that the crystalline complex was present in an anhydrous form.

The X-ray powder diffraction pattern of this batch is shown in Table 6 and depicted in FIG. 6A.

### Preparation of Form 1006:

Step 1: 10 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 1 ml of anisole and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 2): Alternatively, the glassy material from step 1 is mixed with 1 ml of xylene and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 3): The glassy material from step 1 is mixed with 500 μl of cyclopropylmethylether and the resulting suspension is solubilized for 1 h at 60° C. The suspension is cooled by 0.01° C./min down to 5° C. The cooled suspension is filtered using a 0.2 μm filter and dried under vacuum at RT for 1 h.

Similar results were obtained with anisole or toluene as a solvent.

Complementary NMR analysis confirms the chemical integrity of the velagliflozin material, the presence of 1.4 mole eq. of proline, the presence of 1.6 mole eq. of proline and the presence of 1.9 mole eq. of proline, all in the absence of residual solvent.

Several batches of the crystalline complex according to the above preparation are obtained. The X-ray powder diffraction patterns coincide. The melting points are determined via DSC and evaluated as onset-temperature. DSC thermograms show a weak and broad endothermic event between 0° C. and 130° C. associated with the departure of volatile molecules. A large and narrow endothermic peak at ~160° C. is associated with the melting transition of crystalline Form 1006. From these results it could be concluded that the crystalline complex was present in an anhydrous form.

The X-ray powder diffraction pattern as contained in Table 7 and as depicted in FIG. 7A and the DSC and TG diagram in FIG. 7B correspond to a batch with a melting point of approximately 160° C.

### Preparation of Form 1007:

Step 1: 10 mg of crystalline complex (1:1) of velagliflozin with L-proline is dissolved in 500 μl of methanol. The resulting solution is incubated at 40° C. (evaporation under N₂ gas flow) to obtain a glassy material.
Step 2: This material is mixed with 100 μl of ethanol and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 2): Alternatively, the glassy material from step 1 is mixed with 1 ml of xylene and the resulting suspension is solubilized for 1 h at 60° C. The suspension is cooled by 0.01° C./min down to 5° C. The cooled suspension is filtered using a 0.2 μm filter and dried under vacuum at RT for 1 h.
Step 2 (alternative 3): The glassy material from step 1 is mixed with 300 μl of methanol and the resulting suspension is solubilized for 1 h at 60° C. The suspension is cooled by 0.01° C./min down to 5° C. The cooled suspension is filtered using a 0.2 μm filter and dried under vacuum at RT for 1 h.

Obtained solid is stored at least 30 days under room conditions.

Several batches of the crystalline complex according to the above preparation are obtained. The X-ray powder diffraction patterns coincide. The melting points are determined via DSC and evaluated as onset-temperature. From these results it could be concluded that the crystalline complex was present in a hydrated form.

The X-ray powder diffraction pattern as contained in Table 8 and as depicted in FIG. 8A and the DSC and TG diagram in FIG. 8B correspond to a batch with a dehydration noted at around 60° C.

### Preparation of Form 1008:

Step 1: in 2 ml hermetic glass vial, 33 mg of crystalline complex (1:1) of velagliflozin with L-proline is suspended in 100 μl of Water/Tetrahydrofuran 0.02/0.98 (volume ratio) binary mixture.
Step 1 (alternative 2): in 2 ml hermetic glass vial, 56 mg of crystalline complex (1:1) of velagliflozin with L-proline is suspended in 100 μl of Water/Tetrahydrofuran 0.15/0.85 (volume ratio) binary mixture.
Step 2: After 30 days of storage, the suspension is mixed by vortex stirring. The cooled suspension is filtered using a 0.2 μm filter.

Complementary NMR analysis confirms the chemical integrity of the velagliflozin material, the presence of 1 mole eq. of proline and the presence of 0.4 mole eq. of THF.

Several batches of the crystalline complex according to the above preparation are obtained. The X-ray powder diffraction patterns coincide. The melting points are determined via DSC and evaluated as onset-temperature. From these results it could be concluded that the crystalline complex was present in a hydrated/solvated form. The X-ray powder diffraction pattern as contained in Table 9 and as depicted in FIG. 9A and the DSC and TG diagram in FIG. 9B correspond to a batch with a dehydration/desolvation noted around 60° C.

### Preparation of Form 1009:

Step 1: In a 6 ml stainless steel milling tube, 2 stainless steel milling balls of 5 mm diameter are placed.
Step 2: 11.3 mg of Theobromine and 25.3 mg of crystalline complex (1:1) of velagliflozin with L-proline are added into the tube.
Step 3: 9 μl of Methanol are added into the tube, immediately after addition of solvent, the tube is hermetically closed.
Step 4: Immediately after preparation, the tube is placed on the milling instrument (Retsch MM200) for 45 min milling at 20 Hz.

Several batches of the crystalline complex according to the above preparation are obtained. The X-ray powder diffraction patterns coincide. The melting points are determined via DSC and evaluated as onset-temperature. From these results it could be concluded that the crystalline complex was present in an anhydrous form.

The X-ray powder diffraction pattern as contained in Table 10 and as depicted in FIG. 10A and the DSC and TG diagram in FIG. 10B correspond to a batch with a melting point of 120° C.

### REFERENCES

(1) WO 2007/093610
(2) WO 2007/128749
(3) WO 2014/016381
(4) WO 2023/006718

The following clauses are also part of the disclosure and are comprised by the spirit and scope of the present invention:
1. Crystalline complex between velagliflozin and a co-crystal forming agent selected from L-proline, theobromine, 3,5-dihydroxybenzoic acid, maltol, thymol, and caffeine, characterized in that the crystalline complex is a complex of velagliflozin and the co-crystal forming agent with a molar ratio in the range from about 1:1 to about 1:3.
2. Crystalline complex according to clause 1, wherein the co-crystal forming agent is selected from L-proline and theobromine.
3. Crystalline complex according to clause 1 or 2, wherein the co-crystal forming agent is L-proline.
4. Crystalline complex according to clause 1 or 2, wherein the co-crystal forming agent is theobromine.
5. Crystalline complex according to clause 1, wherein the co-crystal forming agent is 3,5-dihydroxybenzoic acid.
6. Crystalline complex according to clause 1, wherein the co-crystal forming agent is maltol.
7. Crystalline complex according to clause 1, wherein the co-crystal forming agent is thymol.
8. Crystalline complex according to clause 1, wherein the co-crystal forming agent is caffeine.
9. Crystalline complex according to any one of clauses 1 to 3, characterized in that the crystalline complex is a complex between velagliflozin and L-proline and in that it has a content of water of about 0 mol, i.e. it is a water-free/anhydrous co-crystal of velagliflozin with L-proline.
10. Crystalline complex according to any one of clauses 1 to 3 and 9 having a content of water of about 0 mol, characterized in that the crystalline complex is a complex (1:3) between velagliflozin and L-proline; and characterized by an X-ray powder diffraction pattern that comprises peaks at 5.77, 16.89, 18.95 and 20.39 degrees 2Θ (±0.1 degrees 2Θ), wherein said X-ray powder diffraction pattern is made using CuK_{α1} radiation.
11. Crystalline complex according to clause 10, wherein the X-ray powder diffraction pattern further comprises peaks at 17.40, 21.59, 23.69 and 25.19 degrees 2Θ (±0.1 degrees 2Θ), wherein said X-ray powder diffraction pattern is made using CuK_{α1} radiation.
12. Crystalline complex according to any one of clauses 10-11, wherein the X-ray powder diffraction pattern further comprises peaks at 5.10, 8.06, 9.64, 18.50, 19.36, 21.10, 23.23 and 26.06 degrees 2Θ (±0.1 degrees 2Θ), wherein said X-ray powder diffraction pattern is made using CuK_{α1} radiation.
13. Crystalline complex according to any one of clauses 10-12, wherein the X-ray powder diffraction pattern further comprises peaks at 11.58, 15.24, 17.88, 19.57, 22.13, 22.76, 24.64, 27.63 and 28.22 degrees 2θ (±0.1 degrees 2Θ), wherein said X-ray powder diffraction pattern is made using CuK_{α1} radiation.
14. Crystalline complex according to any one of clauses 10-13, wherein the X-ray powder diffraction pattern, made using CuK_{α1} radiation comprises peaks at degrees 2Θ (±0.1 degrees 2Θ) as contained in Table 1.
15. Crystalline complex according to any one of clauses 10-14, characterized by an X-ray powder diffraction pattern, made using CuK_{α1} radiation, and comprising peaks at degrees 2Θ (±0.1 degrees 2Θ) as shown in FIG. 1A.
16. Crystalline complex according to any one of clauses 1 to 3 in a hydrate form, characterized in that the crystalline complex is a complex of velagliflozin and L-proline with a molar ratio in the range from about 1:1 to about 1:1.3; and characterized by an X-ray powder diffraction pattern that comprises peaks at 4.00, 6.07, 7.30 and 20.36 degrees 2Θ (±0.1 degrees 2Θ), wherein said X-ray powder diffraction pattern is made using Cuk 1 radiation.
17. Crystalline complex according to clause 16, wherein the X-ray powder diffraction pattern further comprises peaks at 18.29, 18.70, 19.90 and 22.09 degrees 2Θ (±0.1 degrees 2Θ), wherein said X-ray powder diffraction pattern is made using CuK_{α1} radiation.
18. Crystalline complex according to any one of clauses 16-17, wherein the X-ray powder diffraction pattern further comprises peaks at 14.59, 16.84, 17.24, 23.39, 24.49, 25.62, 27.10, and 27.45 degrees 2θ (±0.1 degrees 2Θ), wherein said X-ray powder diffraction pattern is made using CuK_{α1} radiation.
19. Crystalline complex according to any one of clauses 16-18, wherein the X-ray powder diffraction pattern further comprises peaks at 16.15, 22.57, 24.28, 24.74, 29.45, 30.79, 34.97 and 36.18 degrees 2θ (±0.1 degrees 2Θ), wherein said X-ray powder diffraction pattern is made using CuK_{α1} radiation.
20. Crystalline complex according to any one of clauses 16-19, wherein the X-ray powder diffraction pattern, made using CuK_{α1} radiation, which comprises peaks at degrees 2Θ (±0.1 degrees 2Θ) as contained in Table 2.
21. Crystalline complex according to any one of clauses 16-20, characterized by an X-ray powder diffraction pattern, made using CuK_{α1} radiation, which comprises peaks at degrees 2Θ (±0.1 degrees 2Θ)) as shown in FIG. 2A.
22. Crystalline complex according to any one of clauses 1 to 3 in a hydrate form, characterized in that the crystalline complex is a complex (1:1.5) between velagliflozin and L-proline; and characterized by an X-ray powder diffraction pattern that comprises peaks at 7.47, 16.28, 18.87 and 25.84 degrees 2Θ (±0.1 degrees 2Θ), wherein said X-ray powder diffraction pattern is made using CuK_{α1} radiation.
23. Crystalline complex according to clause 22, wherein the X-ray powder diffraction pattern further comprises peaks at 5.47, 10.57, 20.51 and 20.65 degrees 2Θ (±0.1 degrees 2Θ), wherein said X-ray powder diffraction pattern is made using CuK_{α1} radiation.
24. Crystalline complex according to any one of clauses 22-23, wherein the X-ray powder diffraction pattern further comprises peaks at 11.59, 17.20, 17.40, 21.24, 21.63, 21.96, 22.53 and 23.17 degrees 2θ (±0.1 degrees 2Θ), wherein said X-ray powder diffraction pattern is made using CuK_{α1} radiation.
25. Crystalline complex according to any one of clauses 22-24, wherein the X-ray powder diffraction pattern further comprises peaks at 5.75, 10.91, 15.59, 19.37, 19.73, 19.86, 24.25 and 24.89 degrees 2θ (±0.1 degrees 2Θ), wherein said X-ray powder diffraction pattern is made using CuK_{α1} radiation.
26. Crystalline complex according to any one of clauses 22-25, characterized by an X-ray powder diffraction pattern, made using CuK_{α1} radiation, which comprises peaks at degrees 2Θ (±0.1 degrees 2Θ) as contained in Table 3.
27. Crystalline complex according to any one of clauses 22-26, characterized by an X-ray powder diffraction pattern, made using CuK_{α1} radiation, which comprises peaks at degrees 2Θ (±0.1 degrees 2Θ) as shown in FIG. 3A.
28. Crystalline complex according to any one of clauses 1 to 3 in a hydrate form having a content of water of about 1.5 mol per mol of velagliflozin, characterized in that the crystalline complex is a complex (1:1) between velagliflozin and L-proline; and characterized by an X-ray powder diffraction pattern that comprises peaks at 5.49, 14.45, 18.20 and 23.76 degrees 2Θ (±0.1 degrees 2Θ), wherein said X-ray powder diffraction pattern is made using CuK_{α1} radiation.
29. Crystalline complex according to clause 28, wherein the X-ray powder diffraction pattern further comprises peaks at 16.49, 17.29, 22.30 and 23.49 degrees 2Θ (±0.1 degrees 2Θ), wherein said X-ray powder diffraction pattern is made using CuK_{α1} radiation.
30. Crystalline complex according to any one of clauses 28-29, wherein the X-ray powder diffraction pattern further comprises peaks at 11.52, 18.65, 19.43, 19.99, 20.33, 21.35, 23.11 and 26.34 degrees 2θ (±0.1 degrees 2Θ), wherein said X-ray powder diffraction pattern is made using CuK_{α1} radiation.
31. Crystalline complex according to any one of clauses 28-30, wherein the X-ray powder diffraction pattern further comprises peaks at 6.36, 15.43, 19.16, 20.64, 20.99, 22.73, 24.34 and 26.04 degrees 2θ (±0.1 degrees 2Θ), wherein said X-ray powder diffraction pattern is made using CuK_{α1} radiation.
32. Crystalline complex according to any one of clauses 28-31, characterized by an X-ray powder diffraction pattern, made using CuK_{α1} radiation, which comprises peaks at degrees 2Θ (±0.1 degrees 2Θ) as contained in Table 4.
33. Crystalline complex according to any one of clauses 28-32, characterized by an X-ray powder diffraction pattern, made using CuK_{α1} radiation, which comprises peaks at degrees 2Θ (±0.1 degrees 2Θ) as shown in FIG. 4A.
34. Crystalline complex according to any one of clauses 1 to 3 in a hydrate form having a content of water of about 2 mol per mol of velagliflozin, characterized in that the crystalline complex is a complex (1:2) between velagliflozin and L-proline; and characterized by an X-ray powder diffraction pattern that comprises peaks at 5.94, 16.40, 17.95 and 20.28 degrees 2Θ (±0.1 degrees 2Θ), wherein said X-ray powder diffraction pattern is made using CuK_{α1} radiation.
35. Crystalline complex according to clause 34, wherein the X-ray powder diffraction pattern further comprises peaks at 18.49, 19.40, 19.72 and 23.17 degrees 2Θ) (±0.1 degrees 2Θ), wherein said X-ray powder diffraction pattern is made using CuK_{α1} radiation.
36. Crystalline complex according to any one of clauses 34-35, wherein the X-ray powder diffraction pattern further comprises peaks at 16.12, 17.56, 20.59, 22.21, 22.41, 23.67, 26.59 and 27.38 degrees 2θ (±0.1 degrees 2Θ), wherein said X-ray powder diffraction pattern is made using CuK_{α1} radiation.
37. Crystalline complex according to any one of clauses 34-36, wherein the X-ray powder diffraction pattern further comprises peaks at 8.77, 15.24, 19.09, 21.50, 22.82, 24.10, 24.83 and 25.26 degrees 2θ (±0.1 degrees 2Θ), wherein said X-ray powder diffraction pattern is made using CuK_{α1} radiation.
38. Crystalline complex according to any one of clauses 34-37, characterized by an X-ray powder diffraction pattern, made using CuK_{α1} radiation, which comprises peaks at degrees 2Θ (±0.1 degrees 2Θ) as contained in Table 5.
39. Crystalline complex according to any one of clauses 34-38, characterized by an X-ray powder diffraction pattern, made using CuK_{α1} radiation, which comprises peaks at degrees 2Θ (±0.1 degrees 2Θ) as shown in FIG. 5A.
40. Crystalline complex according to any one of clauses 1 to 3 and 9 having a content of water of about 0 mol, characterized in that the crystalline complex is a complex (1:1) between velagliflozin and L-proline; and characterized by an X-ray powder diffraction pattern that comprises peaks at 5.66, 10.81, 17.03 and 17.75 degrees 2Θ (±0.1 degrees 2Θ), wherein said X-ray powder diffraction pattern is made using CuK_{α1} radiation.
41. Crystalline complex according to clause 40, wherein the X-ray powder diffraction pattern further comprises peaks at 6.48, 21.84, 22.00 and 23.82 degrees 2Θ (±0.1 degrees 2Θ), wherein said X-ray powder diffraction pattern is made using CuK_{α1} radiation.
42. Crystalline complex according to any one of clauses 40-41, wherein the X-ray powder diffraction pattern further comprises peaks at 11.36, 14.41, 20.12, 21.67, 23.02, 23.10, 27.09 and 28.03 degrees 2θ (±0.1 degrees 2Θ), wherein said X-ray powder diffraction pattern is made using CuK_{α1} radiation.
43. Crystalline complex according to any one of clauses 40-42, wherein the X-ray powder diffraction pattern further comprises peaks at 16.10, 19.02, 19.83, 25.60, 25.85, 26.00, 27.34 and 28.47 degrees 2θ (±0.1 degrees 2Θ), wherein said X-ray powder diffraction pattern is made using CuK_{α1} radiation.
44. Crystalline complex according to any one of clauses 40-43, characterized by an X-ray powder diffraction pattern, made using CuK_{α1} radiation, which comprises peaks at degrees 2Θ (±0.1 degrees 2Θ) as contained in Table 6.
45. Crystalline complex according to any one of clauses 40-44, characterized by an X-ray powder diffraction pattern, made using CuK_{α1} radiation, which comprises peaks at degrees 2Θ (±0.1 degrees 2Θ)) as shown in FIG. 6A.
46. Crystalline complex according to any one of clauses 1 to 3 and 9 having a content of water of about 0 mol, characterized in that the crystalline complex is a complex of velagliflozin and L-proline with a molar ratio in the range from about 1:1.4 to about 1:1.8; and characterized by an X-ray powder diffraction pattern that comprises peaks at 6.02, 16.64, 18.24 and 20.74 degrees 2Θ (±0.1 degrees 2Θ), wherein said X-ray powder diffraction pattern is made using CuK_{α1} radiation.
47. Crystalline complex according to clause 46, wherein the X-ray powder diffraction pattern further comprises peaks at 9.06, 15.49, 18.51 and 19.44 degrees 2Θ (±0.1 degrees 2Θ), wherein said X-ray powder diffraction pattern is made using CuK_{α1} radiation.
48. Crystalline complex according to any one of clauses 46-47, wherein the X-ray powder diffraction pattern further comprises peaks at 17.79, 19.74, 22.70, 23.62, 24.06, 25.46, 27.03 and 27.88 degrees 2θ (±0.1 degrees 2Θ), wherein said X-ray powder diffraction pattern is made using CuK_{α1} radiation.
49. Crystalline complex according to any one of clauses 46-48, wherein the X-ray powder diffraction pattern further comprises peaks at 12.06, 13.34, 20.21, 22.16, 23.04, 23.25, 24.48 and 27.31 degrees 2θ (±0.1 degrees 2Θ), wherein said X-ray powder diffraction pattern is made using CuK_{α1} radiation.
50. Crystalline complex according to any one of clauses 46-49, characterized by an X-ray powder diffraction pattern, made using CuK_{α1} radiation, which comprises peaks at degrees 2Θ (±0.1 degrees 2Θ) as contained in Table 7.
51. Crystalline complex according to any one of clauses 46-50, characterized by an X-ray powder diffraction pattern, made using CuK_{α1} radiation, which comprises peaks at degrees 2Θ (±0.1 degrees 2Θ) as shown in FIG. 7A.
52. Crystalline complex according to any one of clauses 1 to 3 in a hydrate form, characterized in that the crystalline complex is a complex (1:1) between velagliflozin and L-proline; and characterized by an X-ray powder diffraction pattern that comprises peaks at 8.69, 16.96, 18.12 and 24.02 degrees 2Θ (±0.1 degrees 2Θ), wherein said X-ray powder diffraction pattern is made using CuK_{α1} radiation.
53. Crystalline complex according to clause 52, wherein the X-ray powder diffraction pattern further comprises peaks at 10.78, 17.24, 20.48 and 21.39 degrees 2Θ (±0.1 degrees 2Θ), wherein said X-ray powder diffraction pattern is made using CuK_{α1} radiation.
54. Crystalline complex according to any one of clauses 52-53, wherein the X-ray powder diffraction pattern further comprises peaks at 6.43, 19.42, 20.06, 21.72, 22.39, 23.43, 27.85 and 28.92 degrees 2θ (±0.1 degrees 2Θ), wherein said X-ray powder diffraction pattern is made using CuK_{α1} radiation.
55. Crystalline complex according to any one of clauses 52-54, wherein the X-ray powder diffraction pattern further comprises peaks at 11.45, 14.58, 16.09, 23.12, 26.33, 27.07, 27.36 and 29.46 degrees 2θ (±0.1 degrees 2Θ), wherein said X-ray powder diffraction pattern is made using CuK_{α1} radiation.
56. Crystalline complex according to any one of clauses 52-55, characterized by an X-ray powder diffraction pattern, made using CuK_{α1} radiation, which comprises peaks at degrees 2Θ (±0.1 degrees 2Θ) as contained in Table 8.
57. Crystalline complex according to any one of clauses 52-56, characterized by an X-ray powder diffraction pattern, made using CuK_{α1} radiation, which comprises peaks at degrees 2Θ (±0.1 degrees 2Θ) as shown in FIG. 8A.
58. Crystalline complex according to any one of clauses 1 to 3 characterized by a content of tetrahydrofuran of about 0.4 mol per mol of velagliflozin, characterized in that the crystalline complex is a complex (1:1) between velagliflozin and L-proline; and characterized by an X-ray powder diffraction pattern that comprises peaks at 4.07, 17.21, 20.30 and 23.50 degrees 2Θ (±0.1 degrees 2Θ), wherein said X-ray powder diffraction pattern is made using CuK_{α1} radiation.
59. Crystalline complex according to clause 58, wherein the X-ray powder diffraction pattern further comprises peaks at 16.09, 18.49, 21.42 and 25.71 degrees 2Θ (±0.1 degrees 2Θ), wherein said X-ray powder diffraction pattern is made using CuK_{α1} radiation.
60. Crystalline complex according to any one of clauses 58-59, wherein the X-ray powder diffraction pattern further comprises peaks at 14.74, 18.87, 21.05, 22.00, 23.71, 24.68, 24.86, and 25.24 degrees 2θ (±0.1 degrees 2Θ), wherein said X-ray powder diffraction pattern is made using CuK_{α1} radiation.
61. Crystalline complex according to any one of clauses 58-60, wherein the X-ray powder diffraction pattern further comprises peaks at 4.43, 12.05, 15.30, 16.67, 19.46, 22.63, 26.29 and 26.42 degrees 2θ (±0.1 degrees 2Θ), wherein said X-ray powder diffraction pattern is made using CuK_{α1} radiation.
62. Crystalline complex according to any one of clauses 58-61, the crystalline complex is characterized by an X-ray powder diffraction pattern, made using CuK_{α1} radiation, which comprises peaks at degrees 2θ (±0.1 degrees 2Θ) as contained in Table 9.
63. Crystalline complex according to any one of clauses 58-62, characterized by an X-ray powder diffraction pattern, made using CuK_{α1} radiation, which comprises peaks at degrees 2Θ (±0.1 degrees 2Θ) as shown in FIG. 9A.
64. Crystalline complex according to any one of clauses 1, 2 and 4, characterized in that the crystalline complex is a complex between velagliflozin and theobromine and in that it has a content of water of about 0 mol, i.e. it is a water-free/anhydrous co-crystal of velagliflozin with theobromine.
65. Crystalline complex according to any one of clauses 1, 2, 4 and 64, characterized in that the crystalline complex is a complex (1:1) between velagliflozin and theobromine; and characterized by an X-ray powder diffraction pattern that comprises peaks at 18.22, 20.15, 22.56 and 25.36 degrees 2Θ (±0.1 degrees 2Θ), wherein said X-ray powder diffraction pattern is made using CuK_{α1} radiation.
66. Crystalline complex according to clause 65, wherein the X-ray powder diffraction pattern further comprises peaks at 14.19, 27.01, 34.11 and 38.63 degrees 2Θ (±0.1 degrees 2Θ), wherein said X-ray powder diffraction pattern is made using CuK_{α1} radiation.
67. Crystalline complex according to any one of clauses 65-66, wherein the X-ray powder diffraction pattern further comprises peaks at 30.65, 30.95, 32.27, and 36.89 degrees 2Θ (±0.1 degrees 2Θ), wherein said X-ray powder diffraction pattern is made using CuK_{α1} radiation.
68. Crystalline complex according to any one of clauses 65-67, wherein the X-ray powder diffraction pattern further comprises peaks at 15.23, 32.08, 36.06, 37.54, and 39.82 degrees 2Θ (±0.1 degrees 2Θ), wherein said X-ray powder diffraction pattern is made using CuK_{α1} radiation.
69. Crystalline complex according to any one of clauses 65-68, the crystalline complex is characterized by an X-ray powder diffraction pattern, made using CuK_{α1} radiation, which comprises peaks at degrees 2θ (±0.1 degrees 2Θ) as contained in Table 10.
70. Crystalline complex according to any one of clauses 65-69, characterized by an X-ray powder diffraction pattern, made using CuK_{α1} radiation, which comprises peaks at degrees 2Θ (±0.1 degrees 2Θ)) as shown in FIG. 10A.
71. A mixture of crystalline forms comprising the crystalline complex according to any one of clauses 1 to 70 in admixture with another crystalline form or forms of velagliflozin.
72. The mixture of crystalline forms according to clause 71 comprising at least 50%-weight, preferably at least 90%-weight, more preferably at least 95%-weight of the crystalline complex in accordance with any one of clauses 1 to 70.
73. A pharmaceutical composition comprising one or more crystalline complexes in accordance with any one of clauses 1 to 70 and/or the mixture of crystalline forms according to any one of clauses 71 to 72.
74. Crystalline complex in accordance with any one of clauses 1 to 70 or the mixture of crystalline forms according to any one of clauses 71 to 72 for use as a medicament.
75. Crystalline complex in accordance with any one of clauses 1 to 70 or the mixture of crystalline forms according to any one of clauses 71 to 72 for use in a method of treatment and/or prevention of diseases or conditions, which can be influenced by inhibiting the sodium-dependent glucose cotransporter SGLT, preferably SGLT-2.
76. Crystalline complex in accordance with any one of clauses 1 to 70 or the mixture of crystalline forms according to any one of clauses 71 to 72 for use in a method of inhibition of the sodium-dependent glucose cotransporter SGLT, preferably SGLT-2.
77. A method for making the crystalline complex in accordance with any one of clauses 1 to 70, said method comprising the following steps:
(a) preparing a solution of velagliflozin and a co-crystal forming agent selected from L-proline, theobromine, 3,5-dihydroxybenzoic acid, maltol, thymol, and caffeine, preferably selected from L-proline and theobromine, in a solvent or a mixture of solvents;
(b) storing the solution to precipitate the crystalline complex out of solution;
(c) removing the precipitate from the solution; and
(d) drying the precipitate optionally until any excess of said solvent or mixture of solvents has been removed.

1. Crystalline complex between velagliflozin and a co-crystal forming agent selected from L-proline, theobromine, 3,5-dihydroxybenzoic acid, maltol, thymol, and caffeine, characterized in that the crystalline complex is a complex of velagliflozin and the co-crystal forming agent with a molar ratio in the range from about 1:1 to about 1:3.

2. Crystalline complex according to claim 1, wherein the co-crystal forming agent is selected from L-proline and theobromine.

3. Crystalline complex according to claim 1 or 2, characterized in that the crystalline complex is a complex between velagliflozin and L-proline and in that it has a content of water of about 0 mol, i.e. it is a water-free/anhydrous co-crystal of velagliflozin with L-proline.

4. Crystalline complex according to claim 3, having a content of water of about 0 mol, characterized in that the crystalline complex is a complex (1:3) between velagliflozin and L-proline; and characterized by an X-ray powder diffraction pattern that comprises peaks at 5.77, 16.89, 18.95 and 20.39 degrees 2Θ (±0.1 degrees 2Θ), wherein said X-ray powder diffraction pattern is made using CuK_{α1} radiation.

5. Crystalline complex according to claim 1 or 2 in a hydrate form, characterized in that the crystalline complex is a complex of velagliflozin and L-proline with a molar ratio in the range from about 1:1 to about 1:1.3; and characterized by an X-ray powder diffraction pattern that comprises peaks at 4.00, 6.07, 7.30 and 20.36 degrees 2Θ (±0.1 degrees 2Θ), wherein said X-ray powder diffraction pattern is made using CuK_{α1} radiation.

6. Crystalline complex according to claim 1 or 2 in a hydrate form, characterized in that the crystalline complex is a complex (1:1.5) between velagliflozin and L-proline; and characterized by an X-ray powder diffraction pattern that comprises peaks at 7.47, 16.28, 18.87 and 25.84 degrees 2Θ (±0.1 degrees 2Θ), wherein said X-ray powder diffraction pattern is made using CuK_{α1} radiation.

7. Crystalline complex according to claim 1 or 2 in a hydrate form, having a content of water of about 1.5 mol per mol of velagliflozin, characterized in that the crystalline complex is a complex (1:1) between velagliflozin and L-proline; and characterized by an X-ray powder diffraction pattern that comprises peaks at 5.49, 14.45, 18.20 and 23.76 degrees 2Θ (±0.1 degrees 2Θ), wherein said X-ray powder diffraction pattern is made using CuK_{α1} radiation.

8. Crystalline complex according to claim 1 or 2 in a hydrate form, having a content of water of about 2 mol per mol of velagliflozin, characterized in that the crystalline complex is a complex (1:2) between velagliflozin and L-proline; and characterized by an X-ray powder diffraction pattern that comprises peaks at 5.94, 16.40, 17.95 and 20.28 degrees 2Θ (±0.1 degrees 2Θ), wherein said X-ray powder diffraction pattern is made using CuK_{α1} radiation.

9. Crystalline complex according to any one of claims 1 to 3, having a content of water of about 0 mol, characterized in that the crystalline complex is a complex (1:1) between velagliflozin and L-proline; and characterized by an X-ray powder diffraction pattern that comprises peaks at 5.66, 10.81, 17.03 and 17.75 degrees 2Θ (±0.1 degrees 2Θ), wherein said X-ray powder diffraction pattern is made using CuK_{α1} radiation.

10. Crystalline complex according to any one of claims 1 to 3, having a content of water of about 0 mol, characterized in that the crystalline complex is a complex of velagliflozin and L-proline with a molar ratio in the range from about 1:1.4 to about 1:1.8; and characterized by an X-ray powder diffraction pattern that comprises peaks at 6.02, 16.64, 18.24 and 20.74 degrees 2Θ (±0.1 degrees 2Θ), wherein said X-ray powder diffraction pattern is made using CuK_{α1} radiation.

11. Crystalline complex according to claim 1 or 2 in a hydrate form, characterized in that the crystalline complex is a complex (1:1) between velagliflozin and L-proline; and characterized by an X-ray powder diffraction pattern that comprises peaks at 8.69, 16.96, 18.12 and 24.02 degrees 2Θ (±0.1 degrees 2Θ), wherein said X-ray powder diffraction pattern is made using CuK_{α1} radiation.

12. Crystalline complex according to claim 1 or 2, characterized by a content of tetrahydrofuran of about 0.4 mol per mol of velagliflozin, characterized in that the crystalline complex is a complex (1:1) between velagliflozin and L-proline; and characterized by an X-ray powder diffraction pattern that comprises peaks at 4.07, 17.21, 20.30 and 23.50 degrees 2Θ (±0.1 degrees 2Θ), wherein said X-ray powder diffraction pattern is made using CuK_{α1} radiation.

13. Crystalline complex according to claim 1 or 2, characterized in that the crystalline complex is a complex between velagliflozin and theobromine and in that it has a content of water of about 0 mol, i.e. it is a water-free/anhydrous co-crystal of velagliflozin with theobromine.

14. Crystalline complex according to any one of claims 1, 2 and 13, characterized in that the crystalline complex is a complex (1:1) between velagliflozin and theobromine; and characterized by an X-ray powder diffraction pattern that comprises peaks at 18.22, 20.15, 22.56 and 25.36 degrees 2Θ (±0.1 degrees 2Θ), wherein said X-ray powder diffraction pattern is made using CuK_{α1} radiation.

15. A mixture of crystalline forms comprising the crystalline complex according to any one of claims 1 to 14 in admixture with another crystalline form or forms of velagliflozin.

16. The mixture of crystalline forms according to claim 15 comprising at least 50%-weight, preferably at least 90%-weight, more preferably at least 95%-weight of the crystalline complex in accordance with any one of claims 1 to 14.

17. A pharmaceutical composition comprising one or more crystalline complexes in accordance with any one of claims 1 to 14 and/or the mixture of crystalline forms in accordance with any one of claims 15 to 16.

18. Crystalline complex in accordance with any one of claims 1 to 14 or the mixture of crystalline forms in accordance with any one of claims 15 to 16 for use as a medicament.

19. Crystalline complex in accordance with any one of claims 1 to 14 or the mixture of crystalline forms in accordance with any one of claims 15 to 16 for use in a method of treatment and/or prevention of diseases or conditions, which can be influenced by inhibiting the sodium-dependent glucose cotransporter SGLT, preferably SGLT-2.

20. Crystalline complex in accordance with any one of claims 1 to 14 or the mixture of crystalline forms in accordance with any one of claims 15 to 16 for use in a method of inhibition of the sodium-dependent glucose cotransporter SGLT, preferably SGLT-2.

21. A method for making the crystalline complex in accordance with any one of claims 1 to 12, said method comprising the following steps:
(a) preparing a solution of velagliflozin and a co-crystal forming agent selected from L-proline, theobromine, 3,5-dihydroxybenzoic acid, maltol, thymol, and caffeine, preferably selected from L-proline and theobromine, in a solvent or a mixture of solvents;
(b) storing the solution to precipitate the crystalline complex out of solution;
(c) removing the precipitate from the solution; and
(d) drying the precipitate optionally until any excess of said solvent or mixture of solvents has been removed.
